SEQUENCE LISTING (12 sequences)

SEQ ID NO: 1 - DNA, 20 bp, Artificial Sequence
  Synthetic primer
ctgactataaccacgtaccc

SEQ ID NO: 2 - DNA, 22 bp, Artificial Sequence
  Synthetic primer
agggagaaattaaagaacttgg

SEQ ID NO: 3 - DNA, 19 bp, Artificial Sequence
  Synthetic primer
tgatgatgagggactgttg

SEQ ID NO: 4 - DNA, 20 bp, Artificial Sequence
  Synthetic primer
gagttccttgtcaccatttg

SEQ ID NO: 5 - DNA, 19 bp, Artificial Sequence
  Synthetic primer
ggcagaagaagaaagttgg

SEQ ID NO: 6 - DNA, 20 bp, Artificial Sequence
  Synthetic primer
actactgcagtttgttcaat

SEQ ID NO: 7 - DNA, 19 bp, Artificial Sequence
  Synthetic primer
tgatgatgagggactgttg

SEQ ID NO: 8 - DNA, 19 bp, Artificial Sequence
  Synthetic primer
tgtgtgctcacaggaatag

SEQ ID NO: 9 - DNA, 18 bp, Artificial Sequence
  Synthetic primer
cgtggacaggaagaaact

SEQ ID NO: 10 - DNA, 18 bp, Artificial Sequence
  Synthetic primer
gtgtggatactgaggaag

SEQ ID NO: 11 - DNA, 20 bp, Artificial Sequence
  Synthetic primer
tgaccccctaatctagttgc

SEQ ID NO: 12 - DNA, 20 bp, Artificial Sequence
  Synthetic primer
aacctgtccatgatcttcct

(19) United States
(12) Patent Application Publication
(10) Pub. No.: US 2016/0340742 A1
(43) Pub. Date: Nov. 24, 2016

(54) Method of Identifying Tyrosine Kinase Receptor Rearrangements in Patients

(71) Applicants: MAYO FOUNDATION FOR MEDICAL EDUCATION AND RESEARCH, Rochester, MN (US); TRANSLATIONAL GENOMICS RESEARCH INSTITUTE, Phoenix, AZ (US)

(72) Inventors: Rondell P. Graham, Rochester, MN (US); Mitesh J. Borad, Tempe, AZ (US); Benjamin R. Kipp, Rochester, MN (US); Emily G. Barr Fritcher, Claremont, MN (US); John Carpten, Phoenix, AZ (US); David Craig, Phoenix, AZ (US)

(73) Assignees: Mayo Foundation for Medical Education and Research, Rochester, MN (US); Translational Genomics Research Institute, Phoenix, AZ (US)

(21) Appl. No.: 15/116,471

(22) PCT Filed: Feb. 4, 2015
(86) PCT No.: PCT/US2015/014518

§ 371 Date: Aug. 3, 2016

Related U.S. Application Data
(60) Provisional application No. 61/935,578, filed on Feb. 4, 2014.

(51) Int. Cl. C12Q 1/68

(52) U.S. Cl. CPC C12Q 1/6886; C12Q 1/6841; C12Q 2600/156; C12Q 2600/106

(57) ABSTRACT

The present invention provides a method of characterizing a cancer by obtaining a sample from a subject suspected of having cancer; and determining whether a fibroblast growth factor receptor (FGFR) fusion is present in the sample, wherein the FGFR fusion comprises a FGFR locus, thereby characterizing the cancer based on the presence or absence of the FGFR fusion.

### TECHNICAL FIELD OF THE INVENTION

The present invention relates generally to methods of diagnosing cancer and more specifically to diagnosing and determining the prognosis of cancer patients using a biomarker based on fibroblast growth factor receptors.

### BACKGROUND ART

Without limiting the scope of the invention, its background is described in connection with diagnosing, treating and determining the prognosis of cancer. Cancer is a generic name for a wide range of cellular malignancies characterized by unregulated proliferation, lack of differentiation, and the ability to invade local tissues and metastasize. These neoplastic malignancies affect, with various degrees of prevalence, every tissue and organ in the body. Fibroblast growth factors (FGFs) and their receptors (FGFR) are expressed at increased levels in several tissues and cell lines and overexpression is believed to contribute to the malignant phenotype. FGFs and FGFRs are a highly conserved group of proteins with instrumental roles in angiogenesis, vasculogenesis, and wound healing, as well as tissue patterning and limb formation in embryonic development. FGFs and FGFRs affect cell migration, proliferation, and survival, providing wide-ranging impacts on health and disease.

The FGFR family comprises four major types of receptors, FGFR1, FGFR2, FGFR3, and FGFR4. These receptors are transmembrane proteins having an extracellular domain, a transmembrane domain, and an intracytoplasmic domain. Each of the extracellular domains contains either two or three immunoglobulin (Ig) domains. Transmembrane FGFRs are monomeric tyrosine kinase receptors, activated by dimerization, which occurs at the cell surface in a complex of FGFR dimers, FGF ligands, and heparin glycans or proteoglycans. Extracellular FGFR activation by FGF ligand binding to an FGFR initiates a cascade of signaling events inside the cell, beginning with the receptor tyrosine kinase activity.

For example, U.S. Pat. No. 8,377,636, entitled, “Biological markers predictive of anti-cancer response to kinase inhibitors,” discloses diagnostic and prognostic methods for predicting the effectiveness of treatment of a cancer patient with inhibitors of EGFR kinase, PDGFR kinase, or FGFR kinase. Based on tumors cells having undergone an EMT, while being mesenchymal-like, still express characteristics of both epithelial and mesenchymal cells, and that such cells have altered sensitivity to inhibition by receptor protein-tyrosine kinase inhibitors, in that they have become relatively insensitive to EGFR kinase inhibitors, but have frequently acquired sensitivity to inhibitors of other receptor protein-tyrosine kinases such as PDGFR or FGFR, methods have been devised for determining levels of specific epithelial and mesenchymal biomarkers that identify such “hybrid” tumor cells (e.g. determination of co-expression of vimentin and epithelial keratins), and thus predict the tumor's likely sensitivity to inhibitors of EGFR kinase, PDGFR kinase, or FGFR kinase.

U.S. Pat. No. 7,982,014, entitled, “FGFR3-IIIc fusion proteins,” discloses FGFR fusion proteins, methods of making them, and methods of using them to treat proliferative disorders, including cancers and disorders of angiogenesis. The FGFR fusion molecules can be made in CHO cells and may comprise deletion mutations in the extracellular domains of the FGFRs which improve their stability. These fusion proteins inhibit the growth and viability of cancer cells in vitro and in vivo. The combination of the relatively high affinity of these receptors for their ligand FGFs and the demonstrated ability of these decoy receptors to inhibit tumor growth is an indication of the clinical value of the compositions and methods provided herein.

U.S. Patent Application Publication No. 2013/0345234, entitled, “FGFR and ligands thereof as biomarkers for breast cancer in HR positive subjects,” discloses methods for diagnosing, treating and determining the prognosis of breast cancer HR+ patient, the methods including detecting the amplification of one or more biomarkers comprising a FGFR ligand such as FGF3, FGF4, FGF19, and/or a FGFR, such as for example FGFR1 in a subject; determining an FGFR1 inhibitor for treating the subject based on the amplification of the one or more biomarkers in the subject; administering to the subject in need thereof the FGFR1 inhibitor and using the one or more biomarkers to indicate prognosis of the subject treated with the FGFR1 inhibitor.

### DISCLOSURE OF THE INVENTION

The present invention provides a method of characterizing a cancer by obtaining a sample from a subject suspected of having cancer; and determining whether a fibroblast growth factor receptor (FGFR) fusion is present in the sample, wherein the FGFR fusion comprises a FGFR locus, thereby characterizing the cancer based on the presence or absence of the FGFR fusion.

The present invention provides a method for detecting a fibroblast growth factor receptor (FGFR) translocation event in one or more cancer cells by contacting a sample suspected of comprising one or more cancer cells with a plurality of distinguishably labeled probes capable of hybridizing to a portion of a fibroblast growth factor receptor (FGFR) fusion in the one or more cancer cells; hybridizing a first probe to a first region to form a first hybridization complex; hybridizing a second probe to a second region to form a second hybridization complex; and analyzing the first hybridization complex and the second hybridization complex to identify the presence of a FGFR fusion.

The present invention provides a method for identifying the response of a proliferative disorder responsive to treatment by detecting one or more FGFR biomarkers selected for a FGFR-fusion that is indicative of the prognosis of a subject.

### DESCRIPTION OF THE DRAWINGS

For a more complete understanding of the features and advantages of the present invention, reference is now made to the detailed description of the invention along with the accompanying figures and in which:

FIG. 1A is an image of a tumor that shows intraductal growth and multiple foci with a nested architecture characterized by peripheral cells with scant cytoplasm surrounding cells with more open chromatin and more cytoplasm.

FIG. 1B is an image of the neoplastic cells showed only focal cytokeratin 19 expression.

FIG. 1C is an image of representative photomicrograph of prominent intraductal growth which characterized several cases.

FIG. 1D is an image showing both of these cases revealed FGFR2 translocations using a break-apart FISH probe.

FIG. 2A is an image of a low grade biliary intraductal papillary neoplasm of bile duct forming papillae with complex back to back glands.

FIG. 2B is an image of numerous goblet cells were admixed with the other columnar neoplastic cells.

FIG. 2C is an image of Cytokeratin 19 expression that was diffuse and strong.

FIG. 2D is an image of FGFR2 FISH confirmed translocation of FGFR2.

FIG. 3A is an image of an example of the anastomosing tubular architecture seen in a subset of tumors with FGFR2 translocations.

FIG. 3B is an image as seen in several cases, CK19 expression was patchy and weak.

FIG. 3C is an image of focally, the glands coalesced to form more solid areas.

FIG. 3D is an image of FGFR2 was translocated as confirmed by FISH.

FIG. 4A is an image of FISH showing HER2 amplification.

FIG. 4B is an image of FISH showing ROS1 translocation using break-apart FISH probe.

FIGS. 5A-5G are graphs showing the sequence variation effects.

FIGS. 6A and 6B are representative fluorescent in situ hybridization (FISH) demonstrating the presence of FGFR2 fusion. FIG. 6A shows cholangiocarcinoma with FGFR2 rearrangement.

FIG. 6B shows cholangiocarcinoma negative for FGFR2 rearrangement.

FIG. 7 is an image showing the copy number changes and structural rearrangements.

FIGS. 8A-8B are images of immunohistochemistry demonstrating FGFR2 and FGFR3 expression.

FIGS. 9A-9B are images showing immunohistochemistry demonstrating pFRS2 Y436, and pERK expression in Patients 1, 4, 5, and 6.

FIGS. 10A-10D are images showing transcripts and hypothetical protein products modeled to illustrate the potential functional impact of fusion events involving FGFR2.

### DESCRIPTION OF EMBODIMENTS

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention provides methods of diagnosing, treating and determining the prognosis of a disease or condition comprising abnormal cell growth, the disease or condition comprising abnormal cell growth in one embodiment is a cancer. The present invention is directed to methods for diagnosing, selecting for treatment and determining the prognosis cancer patients using a biomarker based on fibroblast growth factor receptors and determining which patients will most benefit from treatment with inhibitors of receptor protein-tyrosine kinases.

The terms “receptor tyrosine kinase” and “RTK” are used interchangeably herein to refer to the family of membrane receptors that phosphorylate tyrosine residues. Many play significant roles in development or cell division. Receptor tyrosine kinases possess an extracellular ligand binding domain, a transmembrane domain and an intracellular catalytic domain. The extracellular domains bind cytokines, growth factors or other ligands and are generally comprised of one or more identifiable structural motifs, including cysteine-rich regions, fibronectin III-like domains, immunoglobulin-like domains, EGF-like domains, cadherin-like domains, kringle-like domains, Factor VIII-like domains, glycine-rich regions, leucine-rich regions, acidic regions and discoidin-like domains. Activation of the intracellular kinase domain is achieved by ligand binding to the extracellular domain, which induces dimerization of the receptors. A receptor activated in this way is able to autophosphorylate tyrosine residues outside the catalytic domain, facilitating stabilization of the active receptor conformation. The phosphorylated residues also serve as binding sites for proteins which will then transduce signals within the cell. Examples of RTKs include, but are not limited to, Kit receptor (also known as Stem Cell Factor receptor or SCF receptor), fibroblast growth factor (FGF) receptors, hepatocyte growth factor (HGF) receptors, insulin receptor, insulin-like growth factor-1 (IGF-1) receptor, nerve growth factor (NGF) receptor, vascular endothelial growth factor (VEGF) receptors, PDGF-receptor-.alpha., PDGF-receptor-.beta., CSF-1-receptor (also known as M-CSF-receptor or Fms), and the F1t3-receptor (also known as F1k2).

As used herein, the term “subject” is intended to include human and non-human animals. Non-human animals include all vertebrates, e.g. mammals and non-mammals, such as non-human primates, sheep, dogs, cats, cows, horses, chickens, amphibians, and reptiles.

An “FGFR fusion protein” is a protein typically comprising a sequence of amino acids corresponding to the extracellular domain of an FGFR polypeptide or a biologically active fragment thereof, and a fusion partner. The fusion partner may be joined to either the N-terminus or the C-terminus of the FGFR polypeptide and the FGFR may be joined to either the N-terminus or the C-terminus of the fusion partner. An FGFR fusion protein can be a product resulting from splicing strands of recombinant DNA and expressing the hybrid gene. An FGFR fusion protein may comprise a fusion partner comprising amino acid residues that represent some or all of, one or more fragments of, one or more genes. The FGFR fusion molecules of the invention comprise a first polypeptide that comprises an extracellular domain (ECD) of an FGFR polypeptide and a fusion partner. The FGFR polypeptide can be any of FGFR1, FGFR2, FGFR3, and FGFR4, including all their variants and isoforms. Hence, the family of FGFR polypeptides suitable for use in the invention includes FGFR1, FGFR1-IIIb, FGFR1-IIIc, FGFR2, FGFR2-IIIb, FGFR2-IIIc, FGFR3, FGFR3-IIIb, FGFR3-IIIc, FGFR4 and FGFR5, for example. The extracellular domain of the FGFR can be the entire ECD or a portion thereof.

A “fusion partner” is any component of a fusion molecule in addition to the extracellular domain of an FGFR or fragment thereof. A fusion partner may comprise a polypeptide, such as a fragment of an immunoglobulin molecule, or a non-polypeptide moiety, for example, polyethylene glycol. The fusion partner may comprise an oligomerization domain such as an Fc domain of a heavy chain immunoglobulin.

Patients with cholangiocarcinoma often present with locally advanced or metastatic disease. At present, there is a need for more effective traditional chemotherapeutic or targeted therapy strategies to treat patients with cholangiocarcinoma. 152 cholangiocarcinomas and 4 intraductal papillary biliary neoplasms of the bile duct were evaluated for presence of FGFR2 translocations by fluorescence in situ hybridization (FISH) and characterized the clinical, pathologic and immunohistochemical features of cases with FGFR2 translocations. In addition, 100 cholangiocarcinomas were assessed for ERBB2 amplification and ROS1 translocations, of which 3 (3%) and 1 (1%) where positive, respectively. Eight percent (13 of 156) of biliary tumors harbored FGFR2 translocations, including 12 intrahepatic cholangiocarcinomas and 1 intraductal papillary neoplasm of the bile duct. Thirteen percent (12/96) of intrahepatic cholangiocarcinomas harbored a FGFR2 translocation. FGFR2 translocations were also associated with a female predominance, longer disease-free and overall survival, and lack of underlying fibrotic liver disease. Lesions with FGFR2 translocations were frequently associated with weak and patchy expression of CK19. Markers of stem cell phenotype in cholangiocarcinoma, HepPar1 and CK20, were negative in all cases. This is the largest known study of cholangiocarcinomas assessing for FGFR2 translocations and confirms that FGFR2, ERRB2, and ROS1 alterations are potential therapeutic targets in cases of intrahepatic cholangiocarcinoma, with FGFR2 present at the highest frequency.

The present invention provides a fluorescent in situ hybridization (FISH) break-apart assay to detect fusions involving fibroblast growth factor receptor 2 (FGFR2) in patients with cholangiocarcinoma. The assay is able to discern true positive (in 3 of 3 RNA-Seq/Sanger-polymerase chain reaction validated cases) and true negative cases (in 3 of 3 RNA-Seq/Sanger-polymerase chain reaction validated cases). The present invention allows for rapid and reliable detection of cholangiocarcinoma patients with FGFR2 fusions for treatment with fibroblast growth factor receptor inhibitors.

The present invention provides molecular techniques which have led to the identification of therapeutic targets for various tumors, e.g., identified fibroblast growth factor receptor gene (FGFR2) translocations in cholangiocarcinoma which benefited from FGFR targeted therapy. FGFR2 and ROS1 Fluorescence In Situ Hybridization (FISH). Using the hematoxylin and cosin-stained slides as a guide, unstained 5 micron thick glass slides from a selected paraffin block were etched to indicate the areas of tumor for subsequent molecular testing. Slides were placed in an oven at 90° C. for 10 minutes and then pretreated with xylene at room temperature for two consecutive 15 minute intervals. Slides were then immersed in 100% ethanol for 5 minutes and allowed to air dry at 30° C. for 3 minutes. Acid treatment was then performed for 45 minutes using 10mM of citric acid at 80° C. This was followed by SSC pretreatment for 5 minutes at 37° C. and pepsin digestion (0.2%) for 48 minutes. The slides were then dehydrated in serial ethanol baths of increasing concentration and air dried for 5 minutes. FGFR2 break-apart FISH probe (Abbott Molecular Diagnostics, Des Plaines, Ill.) containing Spectrum Orange and Spectrum Green probes were used.

Three to 10 uL of FGFR2 break-apart FISH probe (Abbott Molecular Diagnostics, Des Plaines, Ill.) containing Spectrum Orange and Spectrum Green probes flanking the region of interest was then applied to the etched area of the slide and cover slipped. Hybridization was performed on a HYBRITE™ (Abbott Molecular Inc.) by denaturing at 80° C. for 3 minutes and hybridizing for 12 hours at 37° C. The slides were then removed from the HYBRITE™ and placed in 0.1% NP40/2×SSC at 74° C. for 2 minutes and transferred to a room temperature solution of 0.1% NP40/2×SSC for an additional 2 minutes. DAPI-I counterstain was applied to the sections and the slides were cover slipped.

FISH analyses were then performed in blinded fashion. In order to be considered positive, separate Spectrum Orange and/or Spectrum Green signals were present in greater than 20% of nuclei throughout the tumor. Cases not meeting these criteria were considered negative. All cases with FGFR2 translocation and a subset of cases without translocation were reviewed blindly by a second reviewer. For the ROS-1 break-apart probe, the same method was used. Cholangiocarcinomas (N=3) with FGFR2 translocations and FGFR2 overexpression from global transcriptome sequencing along with cholangiocarcinomas without FGFR2 translocations (N=5) were also evaluated as control specimens in a blinded fashion with this FISH strategy to verify accuracy of the FISH probes.

HER2 Immunohistochemistry and FISH. Five micron unstained sections from the chosen paraffin block were used for HER2 immunohistochemistry using the HercepTest kit (Dako, Carpinteria, Calif.) and following the manufacturer-provided protocol. The slides were reviewed by two pathologists and classified as negative, 1+, 2+ or 3+ based on previously published guidelines by the College of American Pathologists (CAP) and American Society for Clinical Oncologists (ASCO). In all 2+ or 3+ positive cases, the invasive tumor with immunoreactivity was circled and the sections were selected for HER2 FISH.

Immunohistochemistry using commercially available antibodies directed to cytokeratin 7 (clone OV-TL 12/30; 1:200, Dako, Calif.), cytokeratin 19 (clone RCK 108; 1:20,Dako, Calif.), cytokeratin 20 (clone Kₛ 20.8, 1:200, Dako, Calif.), CD56 (clone 123C3; 1:100; Dako, Calif.), KIT (rabbit polyclonal; 1:500; Dako, Calif.) and HepPar 1 (clone OCH1E5; predilute; Ventana, Ariz.) was performed using 30-32 minute pretreatment and standard methods on each of the cases of cholangiocarcinoma with an FGFR2 translocation.

Statistical Analysis. The associations between the occurrence of FGFR2 rearrangements and clinicopathologic results were assessed using JMP 9.0 software and Wilcoxon test and Fisher Exact tests, as appropriate. Kaplan-Meier curves were plotted, and survival was compared using the log-rank value. All reported p values were 2 sided and p values <0.05 were considered significant.

One hundred and fifty-six specimens were evaluated including 152 cholangiocarcinomas and 4 IPNB. Patients ranged from 28 to 83 years of age with a median age of 62 years. The study included 80 males and 76 females. The 152 cholangiocarcinomas in this surgical series were predominantly intrahepatic (n=96; 63%), but also included hilar (n=25; 16%) and extrahepatic (n=31; 20%) tumors. The 4 IPNB specimens included 2 intrahepatic and 2 extrahepatic neoplasms. Two of the IPBN featured low grade dysplasia and the other 2 displayed features of high grade dysplasia. The median maximum dimension of the cholangiocarcinomas was 4.75 cm (range 0.5-14.0 cm) and the median maximum tumor size for the IPNB was 3.15 cm (range 1.8-7.5 cm).

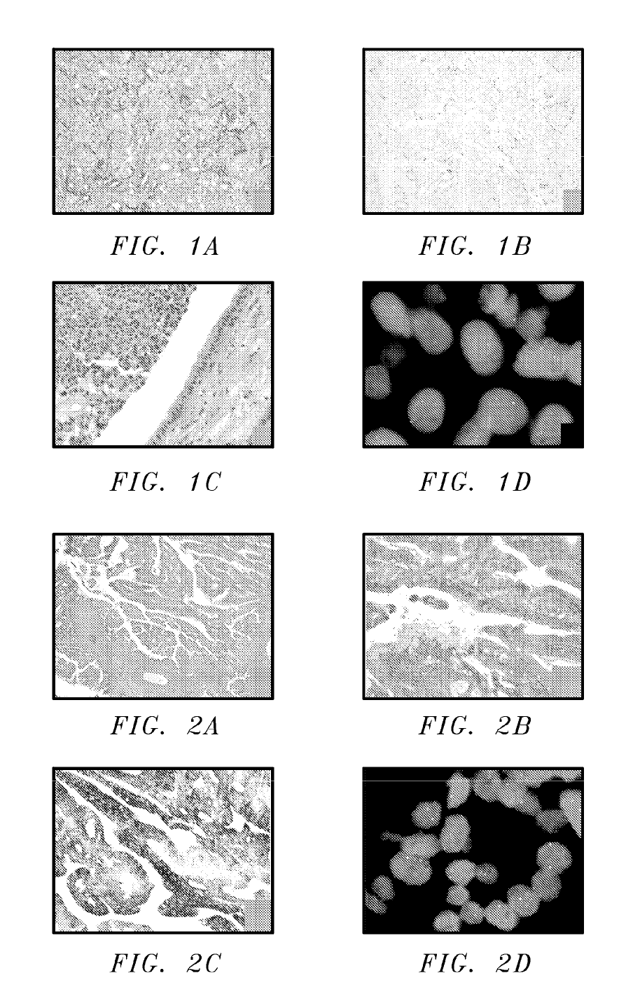

Using FISH, FGFR2 translocations were identified in 12 cholangiocarcinomas and 1 IPNB for an overall frequency of 8% (13/156; FIGS. 1 and 2). All translocated specimens were intrahepatic, with a frequency of 13% (12/96) for intrahepatic cholangiocarcinomas. One of the 13 specimens with an FGFR2 translocation was previously reported as having IDII1mutation. There were significantly (p=0.043) more FGFR2 translocations identified in women (10/76, 13%) than in men (3/80, 4%). The patients with FGFR2-rearranged tumors had a median age of 52 years (range 36-83 years), which was 11 years younger than the median age for the biliary tumors without FGFR2 rearrangement, but this was not significant (p=0.121). The median tumor size for FGFR2 translocated cases was 6.0 cm (range 1.4-13.4 cm) which was similar to the median tumor size for tumors without FGFR2 rearrangement (p=0.201).

Morphologically, cases harboring FGFR2 translocations could be divided into 2 architectural groups; cases (8/13, 62%) which were characterized by prominent intraluminal growth in bile ducts (bile duct invasion/extension) and cases which did not (5/13, 38%). Of the former group, 3 cases were composed predominantly of solid nodules, 4 showed a predominantly trabecular pattern and the other was the case of an intestinal type IPNB. The cases with solid nodules were characterized either by syncytial neoplastic cells with indistinct cell membranes or alternatively by cells with distinct cell membranes. The cases with a trabecular pattern typically featured 2 cell populations including a) a peripheral rim of smaller cells with scant cytoplasm and nuclear hyperchromasia and b) central cells with more cytoplasm, round nuclei and open chromatin. In 2 cases, there were overlapping features including areas with a trabecular growth pattern and a two cell population, and solid areas without the two cell population.

FIG. 1A is an image of a tumor that shows intraductal growth and multiple foci with a nested architecture characterized by peripheral cells with scant cytoplasm surrounding cells with more open chromatin and more cytoplasm (original magnification 200×). FIG. 1B is an image of the neoplastic cells showed only focal cytokeratin 19 expression (original magnification 200×). FIG. 1C is an image of representative photomicrograph of prominent intraductal growth which characterized several cases (original magnification 400×). In this example, there is a solid proliferation of neoplastic cells. FIG. 1D is an image showing both of these cases revealed FGFR2 translocations using a break-apart probe as illustrated by this representative FISH image from the case in FIG. 1A. The tumor cells are aneuploid (>2 copies in each nucleus) but orange and green signals are separated confirming rearrangement of FGFR2.

While the IPNB with FGFR2 translocation showed intraluminal growth by definition, it did not harbor solid nodules or trabeculae but instead was composed of back to back anastomosing tubular glands with abundant goblet cells as shown in FIG. 2A-D.

FIG. 2A is an image of a low grade biliary intraductal papillary neoplasm of bile duct forming papillae with complex back to back glands (original magnification 100×). FIG. 2B is an image of numerous goblet cells were admixed with the other columnar neoplastic cells (original magnification 200×) FIG. 2C is an image of Cytokeratin 19 expression that was diffuse and strong (original magnification 200×). FIG. 2D is an image of FGFR2 FISH confirmed translocation of FGFR2.

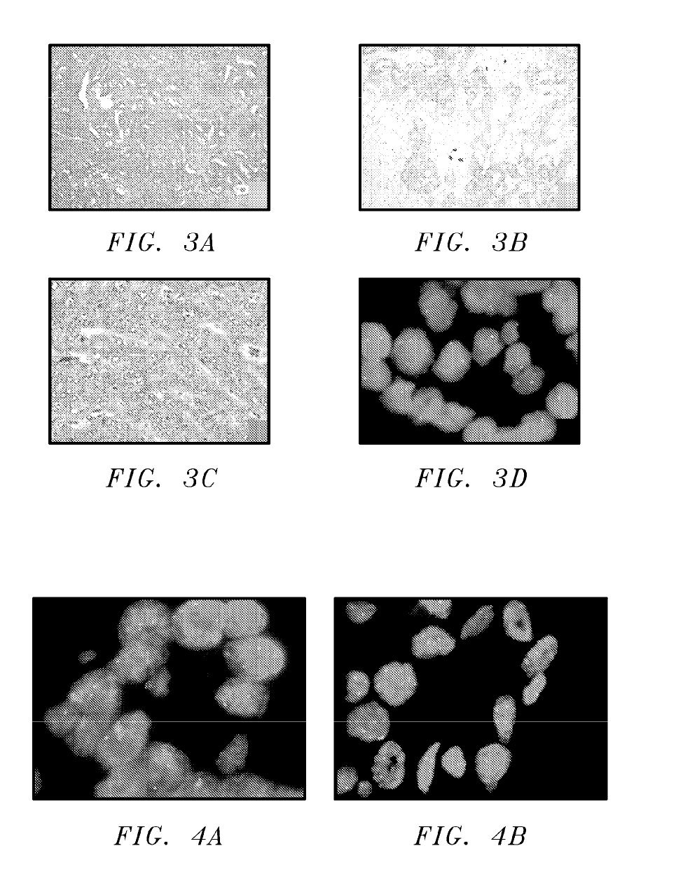

The second group of FGFR2 translocated tumors (5 of 13) included cases which were all composed of anastomosing tubular structures accompanied by desmoplasia. In 2 of the 5 cases, multiple foci of intratubular growth (i.e. glands within a gland) were seen and in the remaining 3 cases the anastomosing tubules coalesced to form solid or syncytial areas in places. FIG. 3A is an image of an example of the anastomosing tubular architecture seen in a subset of tumors with FGFR2 translocations. This was accompanied by an intratumoral neutrophilic infiltrate (original magnification 100×). FIG. 3B is an image as seen in several cases, CK19 expression was patchy and weak (original magnification 200×). FIG. 3C is an image of focally, the glands coalesced to form more solid areas (original magnification 400×). FIG. 3D is an image of FGFR2 was translocated as confirmed by FISH. Separated spectrum orange and green signals are seen confirming translocation of the gene. In these latter 3 cases, there was a prominent intratumoral neutrophilic infiltrate.

By immunohistochemistry, all of the FGFR2-translocated cases were strongly positive for CK7. In 3 cases (23%), CK7 expression was patchy (approximately 10%-50% neoplastic cells positive) while in the remaining 10 cases (77%), CK7 expression was diffuse. Only 3 cases (23%) were diffusely positive for CK19. Six cases (46%) showed weak patchy reactivity for CK19, 3 (23%) revealed focal CK19 expression (<10% neoplastic cells) which was very weak. In a single case, a strong luminal pattern of CK19 expression was seen. Examples of CK19 immunoreactivity are shown in FIGS. 1A-3D. None of the cases expressed CK20, CD56, KIT or HepPar1.

For cholangiocarcinoma cases, clinical follow up was available for 99% (151/152) of patients up to 169 months after surgical resection. For the 139 cases without FGFR2 translocations, 77 patients (55%) developed metastases or local recurrence and 99 patients (71%) died during clinical follow up. Of the 99 patients who died, 69 died of disease (70%), 7 died of other causes (7%) and the cause of death was unknown for 23 patients (23%). The median cancer-specific survival interval for the group without FGFR2 translocations was estimated at 37 months (95% CI: lower limit=24 months, upper limit=49 months) and the median disease free interval was estimated at 26 months (95% CI: lower limit=19 months, upper limit=42 months). Six of the 12 (50%) patients whose tumors harbored FGFR2 translocations died during clinical follow up and 6 patients were alive without evidence of disease. Only 3 patients (25%) developed metastases or local recurrence. The median cancer-specific survival interval for patients with FGFR2 translocations was estimated at 123 months (95% CI: lower limit=51 months, upper limit=123 months) and was significantly longer (p=0.039) than patients without FGFR2 translocations. The disease free intervals for the 3 cases were 26 months, 63 months, and 125 months. Relative to the cases without FGFR2 translocations, this was also significant (p=0.007).

FIG. 4A is an image of FISH showing HER2 amplification (LSI HER-2/neu orange signals)/total CEP 17 green signals>2.2). FIG. 4B is an image of FISH showing ROS1 translocation using break-apart FISH probe. One hundred cases were tested by HER2 immunohistochemistry and 97 were negative. A single 2+ case (intrahepatic) and two 3+ cases (extrahepatic) were identified. FISH confirmed HER2 amplification (HER2/CEP17 ratio >2.2) in each of the 3 cases. None of the HER2 positive cases were positive for FGFR2 translocations. The 3 HER2 positive cases affected 2 men (extrahepatic; ages 69 and 71) and a 46 year old woman (intrahepatic) without PSC or underlying liver disease. The men developed metastatic or recurrent disease and died within 15 months of diagnosis. The woman with the intrahepatic tumor is alive without evidence of recurrence after more than 154 months of follow up.

FISH ROS1 testing was also performed on a group of 100 overlapping cases and was successful in 98 cases. Only a single case revealed a ROS1 translocation, resulting in a rearrangement frequency of 1%. This case was previously reported as harboring an IDH1 mutation. FGFR2 was not translocated in this case. The patient was a 63-year-old woman without underlying liver disease who presented with a localized intrahepatic tumor and is alive with no evidence of disease 66 months after surgery.

Cholangiocarcinoma is a malignancy of the biliary tree and arises within the liver (intrahepatic), at the hilum (central) or within the extrahepatic biliary tree. This anatomic classification is supported in embryology with the extrahepatic bile ducts arising in continuity with the intrahepatic bile ducts but from different cell populations. This classification separates biliary tree malignancies into groups with different mutational spectra and also informs surgical approach. Most cholangiocarcinomas are not amenable to surgical resection at diagnosis and the prognosis is poor. There are currently no FDA-approved targeted therapies for cholangiocarcinoma, a clear unmet clinical need. The present invention provides FISH testing of FGFR2, ERRB2, and ROS1 for the identification of patients whose tumors are candidates for targeted therapies. This is consistent with recent studies suggesting that cholangiocarcinomas harbor mutations that may benefit from tyrosine kinase targeted therapies.

FGFR2, located at chromosome 10q26, is a member of the fibroblast growth factor family of receptors including FGFR1, FGFR3 and FGFR4 and the encoded proteins share highly conserved amino acid sequences. Full length FGFR2, like the other members of the family, is composed of 3 extracellular immunoglobulin domains, an intramembranous segment and a cytoplasmic tyrosine kinase. It interacts with a variety of ligands and the activity of FGFR2 influences proliferation and cellular differentiation. Physiologically, FGFR2 is distributed in ectodermal, endodermal and mesenchymal structures. Point mutations in FGFR2 are associated with congenital craniosynostosis due to abnormal bone development. FGFR2 translocations were identified in a prospective clinical sequencing program in cholangiocarcinoma, breast and prostatic carcinoma. This novel oncogenic mechanism for FGFR2 was validated functionally³⁰ and was subsequently noted by others. As would be expected from their sequence homology, alterations in FGFR1, FGFR3 and FGFR4 have also been demonstrated as oncogenic drivers in various malignancies. Interestingly, prior to the discovery of FGFR2 translocations in cholangiocarcinoma, it was noted that FGFR2 was expressed in 2 cholangiocarcinoma cell lines and that FGFR2 activity not only stimulated neoplastic cell migration but confirmed that inhibition of FGFR2 impaired neoplastic cell migration in the presence of the ligand for FGFR2. Recently, a group identified FGFR2 translocations as a targetable alteration in approximately 15% intrahepatic cholangiocarcinomas which were wild type for KRAS and BRAF and did not harbor ROS1 translocations. FGFR2 is strongly implicated in the development of a subset of cholangiocarcinomas. In this large series of cholangiocarcinomas in patients from the United States, we confirmed the finding of FGFR2 translocations in 13% of intrahepatic tumors. This evidence supports that FGFR2 is a potential therapeutic target in intrahepatic cholangiocarcinoma.

Cholangiocarcinomas with FGFR2 translocations can be grouped morphologically into 2 clusters. The first of which was a group of tumors characterized by intraluminal growth (large duct invasion) by neoplastic cells. Within this group of tumors, 4 tumors formed predominantly solid compact nodules including two cases in which the neoplastic cells were characterized by indistinct cell borders, appeared syncytial in growth pattern and were accompanied by a neutrophilic infiltrate; 3 formed a nested architecture also including a case with a syncytial appearance to the neoplastic cells and were composed of a dual population of cells including a peripheral rim of cells with hyperchromasia and there was a single case of an intraductal papillary biliary neoplasm. The second group of cases (5 of 13) did not reveal large duct invasion and were characterized by anastomosing tubular structures with variable architectural complexity accompanied by a desmoplastic stroma and in 3 of these cases, a prominent neutrophilic infiltrate. It is not clear whether there are biological differences between tumors from these 2 morphologic groups. None of the described features could be used to distinguish cases harboring FGFR2 translocations from cases without FGFR2 translocations.

For some tumor types, morphologic characteristics may be suggestive of underlying molecular alterations. This is illustrated by the presence of abundant tumor infiltrating lymphocytes, signet ring cells and mucinous histology in microsatellite unstable colorectal carcinoma. High grade, triple negative, basal-like breast carcinomas are frequently poorly differentiated with a syncytial pattern of growth and abundant necrosis. Predominantly solid histology has been shown in KRAS mutated lung adenocarcinomas and others have recognized a distinctive recurrent morphologic constellation of features including chromophobe cytoplasm, abrupt anaplasia and pseudocysts in hepatocellular carcinomas with an unusual molecular cytogenetic phenotype. However, none of these morphologic features is sufficiently specific to act as a sole marker for the molecular alterations in routine practice.

Interestingly, a subset of the FGFR2-rearranged cases display stem-cell like features. Together with the physiologic role of FGFR2 in stem cell differentiation in organogenesis, this raised the possibility that tumors with FGFR2 translocations may display a stem cell phenotype. However, the markers of stem cell phenotype in cholangiocarcinomas, CD56 and KIT, were negative. While this argues against a stem cell phenotype, it is worth pointing out that the search for novel markers of stem cells in liver tumors is ongoing and additional robust markers are still needed. Importantly though, 69% (9 of 13) of cholangiocarcinomas harboring FGFR2 translocations showed significantly diminished expression of CK19 suggesting that they were primitive. CK19 is expressed in hepatic progenitor cells in early embryogenesis. At 10 weeks gestation, the expression of CK19 is downregulated in hepatocytes but continues in intrahepatic and extrahepatic bile ducts. This forms the biological basis for CK19 as a marker of pancreatobiliary tumors. Several large studies have reported that CK19 is diffusely positive in 80-100% of cholangiocarcinomas. Therefore, only focal and weak CK19 expression in most of the cases with FGFR2 translocations suggests that this subset of cholangiocarcinomas is enriched for tumors with primitive characteristics. This is also supported by the fact that most tumors revealed solid, syncytial or trabecular growth. Taken together, our data suggests that FGFR2 translocations are associated with intrahepatic neoplasms which display a duct invasive or weakly duct-forming phenotype with predominantly primitive morphologic features.

Both cholangiocarcinomas and hepatocellular carcinomas may arise in the setting of underlying disease or in apparently normal livers. 102 cholangiocarcinomas, 149 colorectal carcinomas, 212 gastric carcinomas and almost 100 hepatocellular carcinomas have been studied for FGFR2 translocations by RT-PCR. They found 5 of 11 total cases (including 1 colorectal carcinoma and 1 hepatocellular carcinoma) with FGFR2 translocations occurred in patients with viral hepatitis B or C. They do not comment on the features of the background liver in the 9 cholangiocarcinoma cases but they compare the rate of viral hepatitis in cases with FGFR2 translocations to control cases and found a statistically significant increase in the rate of viral hepatitis carriage. Two of 16 tested patients were positive for viral hepatitis C and only one of these was associated with fibrosis of the background liver. There were no cases with cirrhosis or primary sclerosing cholangitis. From these data, it is not clear if underlying liver disease or viral hepatitis are important contributors to the pathogenesis of FGFR2 translocation-associated cholangiocarcinomas.

The median disease free and cancer-specific survivals of cases without FGFR2 translocation were 26 and 37 months, respectively, compared to 125 and 123 months respectively for the patients whose tumors harbored FGFR2 translocations. In retrospective studies of patients treated with various combinations of therapy and not matched for performance status and other characteristics, it is difficult to determine the generalizability of survival data. The younger patients, feasibility of resection, unique tumor biology and lack of underlying liver disease may have contributed to the improved survival of patients whose tumors harbored FGFR2 translocations.

Clinically, detecting FGFR2 translocations is relevant because this appears to represent a targetable alteration. Wu et al. identified FGFR2 fusions in 2 cholangiocarcinoma specimens. Arai et al. showed the functional significance of FGFR2 translocations in cholangiocarcinoma including activation of the MAPK pathway and also provided data that FGFR2 inhibition led to diminished MAPK pathway activity. They also performed studies in murine avatars confirming the tractability of FGFR2 translocations in cholangiocarcinomas. If the studies are summed, 27 of 287 biliary neoplasms have been found to harbor FGFR2 translocations which comprise approximately 10% of cases studied and include exclusively intrahepatic tumors. In addition, rare cases with HER2 amplification and ROS1 translocation were identified. These targetable alterations were mutually exclusive of FGFR2 translocation and their molecular biology has already been characterized. FGFR2 translocations in intrahepatic cholangiocarcinoma are associated with a primitive phenotype, apparent female predominance, apparent tendency to longer disease free and overall survival and lack of underlying fibrotic liver disease. As such, FISH testing may be a useful clinical test for the detection of tyrosine kinase receptor rearrangements in patients with cholangiocarcinoma. Lastly, increasing data suggests that targeted therapy for FGFR2, ERBB2 and ROS1 chromosomal alterations are exciting potential treatments for this group of patients who currently have an overall unfavorable prognosis.

For example, advanced cholangiocarcinoma continues to harbor a difficult prognosis and limited therapeutic options. For example, biliary tract cancers (BTC) comprise malignant tumors of the intrahepatic and extrahepatic bile ducts. Known risk factors for BTC are the liver flukes O. viverrini and C. sinensis in high prevalence endemic regions in southeast Asia [1-3], as well as primary sclerosing cholangitis [4-7], Caroli's disease [8], hepatitis B and hepatitis C [9-14], obesity [13], hepatolithiasis [15,16] and thorotrast contrast exposure [17,18]. Surgical approaches such as resection and liver transplantation represent the only curative treatment approaches for BTC [19]. Unfortunately, most patients present with surgically unresectable and/or metastatic disease at diagnosis. Systemic therapy with gemcitabine and cisplatin has been established as the standard of care for patients with advanced disease, but is only palliative, [20] emphasizing the imminent need for novel therapies.

Mutations/allelic loss of known cancer genes in BTC [21-39] have been reported and recently, a prevalence set of 46 patients was used to validate 15 of these genes including: TP53, KRAS, CDKN2A and SMAD4 as well as MLL3, ROBO2, RNF43, GNAS, PEG3, XIRP2, PTEN, RADIL, NCD80, LAMA2 and PCDHA13. Recurrent mutations in IDH1 (codon 132) and IDH2 (codons 140 and 172) have been reported with a prevalence of 22-23% associated with clear cell/poorly differentiated histology and intrahepatic primary [40,41]. Fusions with oncogenic potential involving the kinase gene ROS1 have been identified in patients with BTC with a prevalence of 8.7% [42]. Less frequently, mutations in sporadic BTC have been reported in EGFR [43,44], BRAF [45], NRAS [40,46], PIK3CA [40,46,47], APC [40], CTNNB1 [40], AKT1 [40], PTEN [40], ABCB4 [48], ABCB11 [49,50], and CDH1 [51] as well as amplifications in ERRB2 [52]. FGFR fusions have also been seen in cholangiocarcinoma, e.g. a single case with FGFR2-AHCYL1 [53] as well as several cases identifying FGFR2-BICC1 fusions [53,54]. For example, FGFR2 fusions in a cohort of 102 cholangiocarcinoma patients showed that the fusions occurred exclusively in the intrahepatic cases with a prevalence of 13.6% [53]. Overexpression of the FGFR2-BICC1 and other selected fusions resulted in altered cell morphology and increased cell proliferation [54]. These data led to the conclusion that the fusion partners are facilitating oligomerization, resulting in FGFR kinase activation in tumors possessing FGFR fusions. In addition, in vitro and in vivo assessment of the sensitivity of cell lines containing an FGFR2 fusion to a FGFR inhibitor demonstrated sensitivity to treatment only in the fusion containing cells [53,54], suggesting the presence of FGFR fusions may be a useful predictor of tumor response to FGFR inhibitors.

To comprehensively evaluate the genetic basis of sporadic intrahepatic cholangiocarcinoma (SIC), with emphasis on elucidation of therapeutically relevant targets, integrated whole genome and whole transcriptome analyses was performed on tumors from 6 patients with advanced, sporadic intrahepatic cholangiocarcinoma (SIC). Notably, recurrent fusions involving the oncogene FGFR2 (n=3) were identified.

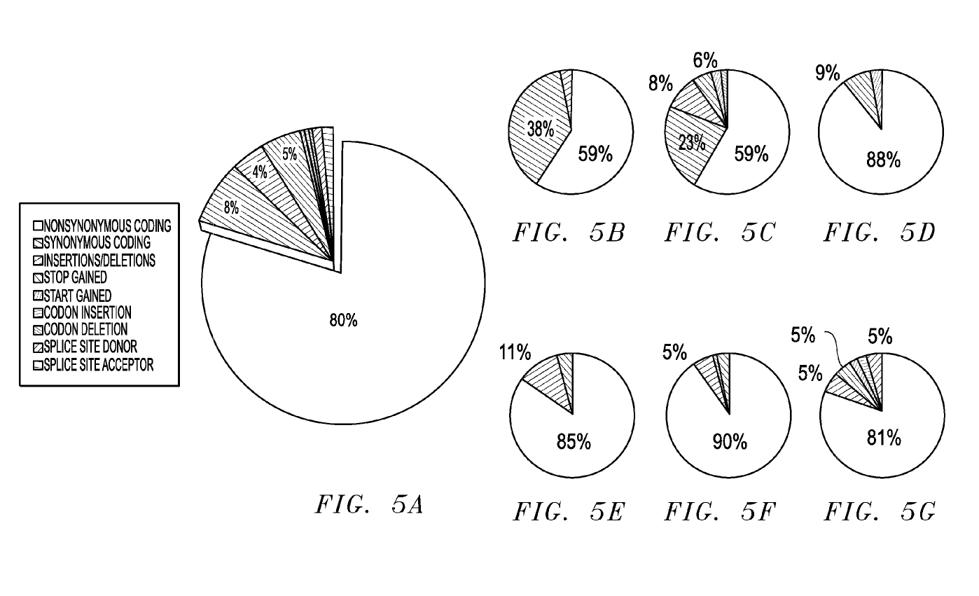

FIGS. 5A-5G are graphs showing the sequence variation effects. Functional effects of high confidence sequence variations for all of the patients were identified. The abundance of variations in each functional category is provided as percentages relative to the total number of high confidence variations and raw counts are provided in Table 1.

TABLE 1
Summary of mutation type by patient

| | Patient 1 | Patient 2 | Patient 3 | Patient 4 | Patient 5 | Patient 6 |
|---|---|---|---|---|---|---|
| Nonsynonymous coding | 20 | 30 | 31 | 44 | 101 | 34 |
| Synonymous coding | 13 | 12 | 0 | 0 | 0 | 0 |
| Insertions/deletions | 1 | 4 | 0 | 6 | 0 | 2 |
| Stop gained | 0 | 3 | 3 | 2 | 6 | 2 |
| Start gained | 0 | 1 | 0 | 0 | 0 | 0 |
| Codon insertion | 0 | 1 | 0 | 0 | 0 | 1 |
| Codon deletion | 0 | 0 | 0 | 0 | 0 | 1 |
| Splice site donor | 0 | 0 | 1 | 0 | 1 | 2 |
| Splice site acceptor | 0 | 0 | 0 | 0 | 4 | 0 |
| Total | 34 | 51 | 35 | 52 | 112 | 42 |

For categories where the percentage was less than 5%, values are not shown.

FIG. 5A shows a summary of the mutation type. Summaries by individual patients are shown in FIG. 5B for Patient 1, FIG. 5C for Patient 2, FIG. 5D for Patient 3, FIG. 5E for Patient 4, FIG. 5F for Patient 5, and FIG. 5G for Patient 6. Nonsynonymous single nucleotide variations were the predominant class in all of the patients. Two patients, Patients 1 and 2 also accumulated a high number of synonymous mutations in comparison to the other patients; Patient 5 carries the most stops gained likely contributing to a higher number of pseudogenes in comparison to the others; Patient 5 was also the only patient to carry several predicted high impact mutations that affect the splice site acceptor regions (light green, percentage <5%). In addition to the major functional classes summarized, Patient 6 also carried a codon change plus insertion variation. A total of 327 somatic coding mutations were identified with an average of 55 mutations/tumor (range 34-112), within our cohort. Nonsynonymous single nucleotide variations were the predominant class in all of the patients. Patients 1 and 2 accumulated a high number of synonymous mutations in comparison to the other patients. Patient 5 carried the most stops gained likely contributing to a higher number of pseudogenes in comparison to the others and was also the only patient to carry several predicted high impact mutations affecting splice site acceptor regions (FIGS. 5A-5G, light green, percentage <5%). In addition, patient 6 also carried a codon change plus insertion variation. Sequencing statistics are provided in Table 2.

TABLE 2
Sequencing metrics of 6 advanced, sporadic biliary tract cancer patients

| | | Exome | | | | Whole Genome | | | RNA Seq | |
|---|---|---|---|---|---|---|---|---|---|---|
| | | Aligned | Mean | | | Aligned | Aligned | | Aligned | Aligned |
| | | Reads | Target | % Target | # of Functional | Reads | Bases | Physical | Reads | Bases |
| Patient | Tissue | (Millions) | Coverage | Bases 10× | Coding Variants | (Millions) | (Billions) | Coverage | (Millions) | (Billions) |
| 1 | N | 161 | 100 | 94% | — | 266 | 22 | 37 | — | — |
| | T | 156 | 112 | 94% | 21 | 228 | 18 | 35 | 100 | 8.1 |
| 2 | N | 176 | 74 | 94% | — | 179 | 14 | 5 | — | — |
| | T | 202 | 81 | 94% | 34 | 370 | 30 | 10 | 341 | 26 |
| 3 | N | 226 | 110 | 58% | — | 296 | 24 | 50 | 163 | 13 |
| | T | 195 | 92 | 58% | 52 | 321 | 26 | 50 | 101 | 8.1 |
| 4 | N | 167 | 80 | 95% | — | 317 | 26 | 42 | — | — |
| | T | 202 | 93 | 96% | 52 | 163 | 13 | 12 | 264 | 20 |
| 5 | N | 257 | 146 | 96% | — | 335 | 27 | 51 | — | — |
| | T | 133 | 78 | 93% | 250 | 349 | 28 | 39 | 401 | 31 |
| 6 | N | 350 | 243 | 92% | — | — | — | — | — | — |
| | T | 340 | 245 | 92% | 43 | — | — | — | 713 | 31 |
| Liver | — | — | — | — | — | — | — | — | 118 | 9.6 |
| Control | | | | | | | | | | |

N = Normal,
T = Tumor

Table 3 (submitted on CD and incorporated herein) is a table of the somatic point mutations, insertions and deletions identified in all samples. Genes with mutations in more than one case included CSPG4 (n=2), GRIN3A (n=2) and PLXBN3 (n=2); with half of these predicted to be potentially damaging by SIFT [55], Polyphen [56], Mutation Assessor [57] and Mutation Taster [58]. While there was overlap in the somatic landscape of SIC with liver-fluke associated cholangiocarcinoma, hepatocellular cancer and pancreatic cancer, most of the aberrations detected in our study were distinct. Table 4 is a comparison of mutation frequency in cholangiocarcinoma, pancreatic and liver caners.

TABLE 4

| | | Liver fluke | | | |
|---|---|---|---|---|---|
| | Non-liver | associated | | | |
| | fluke CCA | CCA [111] | CCA [40] | PDAC [112] | HCC [113] |
| Gene | (n = 6) | (n = 54) | (n = 62) | (n = 142) | (n = 149) |
| AKT1 | 0% | 0% | 1.6% | 0% | 0% |
| APC | 0% | 0% | 0% | 0% | 1.3% |
| ARID2 | 0% | 0% | NA | 2.1% | 6.0% |
| BAP1 | 16.7% | 0% | NA | 0% | 0% |
| BRAF | 0% | 0% | 1.6% | 0.7% | 0% |
| CDKN2A | 0% | 5.6% | NA | 2.4% | 7.4% |
| CSPG4 | 33.3% | 0% | NA | 0% | 0.7% |
| CTNNB1 | 0% | 0% | NA | 0% | 34.9% |
| DMXL1 | 0% | 0% | NA | 0% | 0% |
| EGFR | 0% | 0% | 0% | 0% | 0% |
| ERRFI1 | 16.7% | 0% | NA | 0% | 0.7% |
| FLT3 | 0% | 0% | 0% | 0% | 0% |
| GNAS | 0% | 9.3% | NA | 0.7% | 0% |
| GRIN3A | 33.3% | 0% | NA | 0% | 0% |
| IDH1 | 0% | 0% | 13% | 0% | 0% |
| IDH2 | 16.7% | 0% | 2% | 0% | 0% |
| JAK2 | 0% | 0% | 0% | 0% | 0% |
| KIT | 0% | 0% | 0% | 0% | 0% |
| KRAS | 0% | 16.7% | NA | 66.2% | 1.3% |
| LAMA2 | 16.7% | 3.7% | NA | 0% | 0% |
| MLL3 | 16.7% | 14.8% | NA | 4.9% | 0% |
| NDC80 | 0% | 3.7% | NA | 0% | 0% |
| NLRP1 | 16.7% | 0% | NA | 0% | 0% |
| NOTCH1 | 16.7% | 0% | 0% | 0% | 0% |
| NRAS | 16.7% | 0% | 3.2% | 0% | 0% |
| PCDHA13 | 16.7% | 3.7% | NA | 0.7% | 0% |
| PAK1 | 16.7% | 0% | NA | 0% | 0% |
| PEG3 | 0% | 5.6% | NA | 1.4% | 0% |
| PIK3CA | 0% | 0% | 0% | 0% | 1.3% |
| PLXNB3 | 33.3% | 0% | NA | 0% | 0% |
| PTEN | 0% | 3.7% | 2% | 0% | 0% |
| PTK2 | 16.7% | 0% | NA | 0% | 0% |
| RADIL | 0% | 3.7% | NA | 0% | 0% |
| RNF43 | 0% | 9.3% | NA | 0% | 0% |
| ROBO2 | 0% | 9.3% | NA | 1.4% | 0% |
| SMAD4 | 0% | 16.7% | NA | 11.3% | 0% |
| TP53 | 33.3% | 44.4% | 8% | 23.2% | 19.5% |
| XIRP2 | 0% | 5.6% | NA | 3.5% | 0% |

CCA, cholangiocarcinoma;
PDAC, pancreatic ductal adenocarcinoma;
HCC, hepatocellular carcinoma;
NA, not assessed

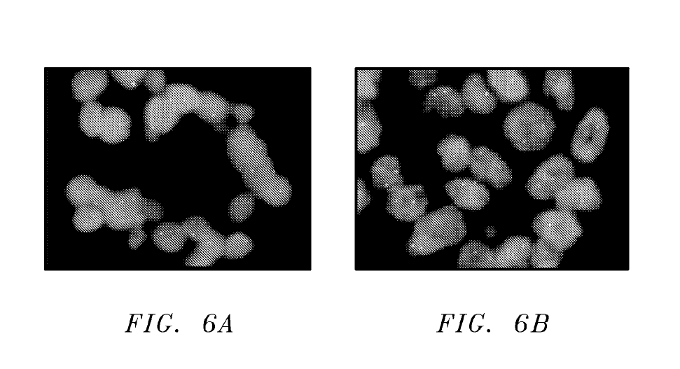

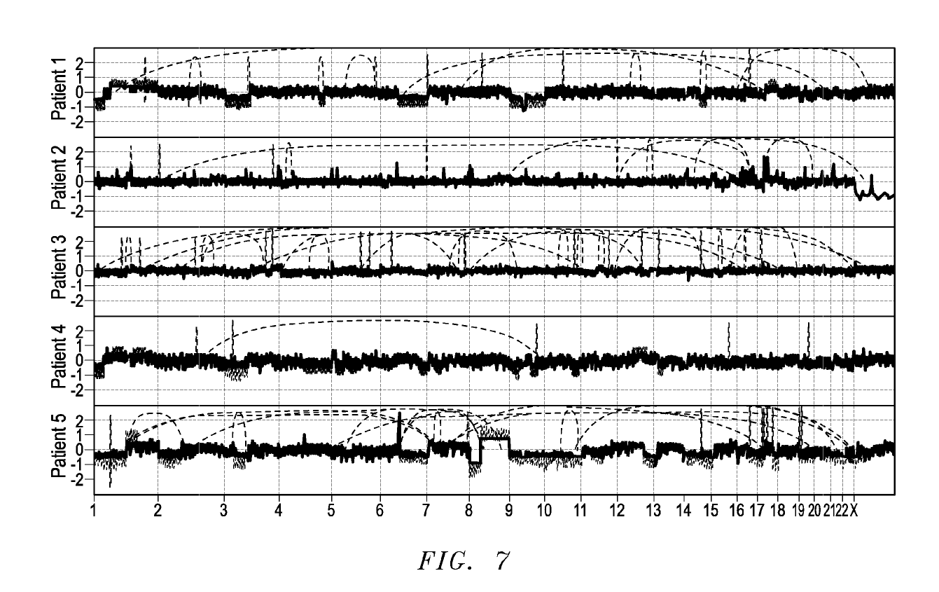

FIGS. 6A-6B are images of representative fluorescent in situ hybridization (FISH) demonstrating the presence of FGFR2 fusion. The present invention provides molecular fusions involving FGFR2 that were therapeutically relevant in 3 patients and were identified with a break apart Fluorescent In situ Hybridization (FISH) assay as seen in FIGS. 6A and 6B. FIG. 6A shows cholangiocarcinoma with FGFR2 rearrangement (distinct orange and green signals are present in most of the cells). FIG. 6B shows Cholangiocarcinoma negative for FGFR2 rearrangement (orange and green signals remain fused). Notably, the patients who did not harbor the FGFR2 fusions were negative using the same assay.

BAP1 (R60*) presented with a truncating mutation that has been previously reported in skin, but have not been reported in biliary cancers. Somatic BAP1 mutations have been identified in a number of tumor types including: Breast, endometrium, eye, kidney, large intestine, lung, ovary, pleura, prostate, skin, and urinary tract. A deubiquitinating enzyme and possible tumor suppressor, BAP1, plays a critical role in the regulation of chromatin modulation and transcription. Furthermore, the loss of BAP1 has been associated with tamoxifen resistance in breast cancer, aggressive and metastatic disease in uveal melanomas.

A nonsynonymous mutation observed in PTK2 (P926S) occurs in a region of the gene whose protein product interacts with TGFB1I1 and ARHGEF28. PTK2, also known as focal adhesion kinase (FAK), is a tyrosine kinase involved in the regulation of cell migration, proliferation, adhesion, microtubule stabilization and actin cytoskeleton. Furthermore, FAK interacts with multiple signaling molecules and in multiple pathways suggesting the possible use of therapeutic treatments directly targeting these interactions or targeting downstream targets of PTK2 such as PI3K or mTOR.

A serine/threonine p21 protein-activated kinase 1 (PAK1) gene contains a nonsynonymous (R371C) mutation located in the protein kinase domain. The location of this mutation could potentially lead to loss of the critical protein kinase domain. While PAK1 is expressed in many normal tissues, it is highly-expressed in ovarian, breast, and bladder cancers. PAK1 plays a role in cell motility, proliferation, survival, and death although, the ability to therapeutically target PAK1 will require further study by tumor type as breast cancer subpopulations have shown response to PAK1 inhibition while non-small cell lung cancer has proven resistant. K5-rTA::tet-KRASG12D mice wildtype for Pak1, responded to treatment with PAK or MEK inhibitors, but did not respond to AKT inhibitors.

Tables 5 (submitted on CD and incorporated herein) 6 and 7 attached hereto are tables showing genes carrying single nucleotide or frameshift variations, or aberrant in copy number were annotated and clustered by GO term functional classes, some of which are known to play a role in Cancer. Proteins predicted to be integral to the membrane and involved in transport, as well as transcriptional regulators were among the most abundant class in all of the patients affected by small scale sequence variations and copy number variations. Variations specifically affecting the EGFR or FGFR gene families were prevalent in Patients 4, 5, and 6 and are highlighted in the figure with the gene name provided in parenthesis next to the pathway name. Comparative pathway analysis of genes carrying small scale nucleotide variations (SsNVs) has implicated several major pathways, possibly interacting as a network, that are predicted to underlie disease in biliary carcinoma patients. These shared pathways include EGFR, EPHB, PDGFR-beta, Netrin-mediated and Beta 1 integrin mediated signaling pathways. Interestingly, most of these pathways have known roles in mediating epithelial-to-mesenchymal cell transitions, which occur frequently during development as well as tumorigenesis. Cell growth and motility is inherent to the successful progression of both biological processes. Studies of the nervous system and lung development have shown that Netrins act to inhibit FGF7 and FGF10 mediated growth or cell guidance [60]. In addition, Netrin-1 has a known role in mediating cell migration during pancreatic organogenesis [60]. Furthermore, Netrin-1 acts as a ligand for α3β1 and α6β4 integrins, both of which are involved in supporting adhesion of developing pancreatic epithelial cells with Netrin-1 although it is thought that α6β4 plays the principle role during this process [60]. Interestingly, α3β1 has been hypothesized to play a role during the process of angiogenesis, when chemoattractants and chemorepellents act to guide filopodia during migration [60]. The α3β1 integrin receptor may act together with additional pathways proposed to play a role during angiogenesis such as VEGF, PDGFR-beta [61], and EphrinB [62] as well as tumorigenesis [60]. Patients 3 and 4 also shared several genes acting in cadherin signaling pathways (see Tables 6-7 submitted on CD and incorporated herein), which are important for maintaining cell-cell adhesion and are known to be intimately integrated with EGFR and FGFR signaling pathways [63].

FIG. 7 is an image showing the copy number changes and structural rearrangements. Whole genome data was utilized to determine copy number alterations and structural rearrangements in the genome for Patients 1-5. WGS was not conducted for patient 6. Red indicates copy number gain, green copy number loss and blue lines indicate structural rearrangements. Significant variability between samples was observed for both copy number changes and structural rearrangements. Patient 5 presented with numerous copy number changes and structural rearrangements contrasting with patient 4 who had minimal structural rearrangements and much smaller regions of copy number changes. Patient 3 is characterized by a large number of structural rearrangements with almost no copy number alterations; in contrast, Patient 1 has a moderate number of structural variations, but has large regions of copy number gain and loss. Patient 2 has a moderate number of structural rearrangements with multiple focal amplifications across the genome.

In addition to the variations identified in genes acting in EGFR, and/or FGFR signaling pathways, multiple sSNVs, and copy number variations (CNVs) (FIG. 7) in genes such as IIDAC1, TP53, MDM2 and AKT1, acting in interaction networks or regulatory pathways involving the fusion partner genes in Patients 5 (BICC1), and 6 (TACC3) (Table 8) (submitted on CD and incorporated herein) are seen. Known mutations in BICC1 have been shown to disrupt canonical Wnt signaling [64] and genes, such as BCL9, involved in this pathway are known to regulate a range of biological processes such as transcription and cell proliferation and carry variations in Patient 5 (Table 8). CSPG4, a target that is being investigated for antibody-based immunotherapy in preclinical studies of triple negative breast cancer [65], is involved in the Wnt signaling pathway, and carries variations in both Patients 1 and 2, however, it is not mutated in Patient 5. TACC3 is known to mediate central spindle assembly and multiple genes including CDCA8, BUB1, and TACC1, belonging to the TACC3 interaction network exhibit aberrant copy number in Patient 6 (Table 8). A recent study has also implicated TACC3 in EGF-mediated EMT when overexpressed [64], and we find that the PLCG1, MAP2K1, and MAPK8 genes, which act in both FGFR and EGFR regulatory pathways, exhibit CNV in Patient 6. The DNAH5 gene encoding a dynein protein is part of the microtubule-associated motor protein complex carries two G→C missense mutations in Patient 6 (Table 4). Several genes carrying more than one variation in either the same patient or different patients also included genes with known roles similar to genes in FGFR/EGFR pathways including axon guidance, invasive growth, or cell differentiation (NAV3, LAMC3, PLXNB3, and PTPRK) (Table 4). In the case of Patient 4, our studies suggest that the primary effect of the FGFR2-MGEA5 fusion is on FGFR2 related signaling, since changes in expression were observed in FGF8 (p<0.05) and the genome of this patient also carries a 4-bp insertion (\hat{0}GTGT) in the FGFR4 gene (Table 4).

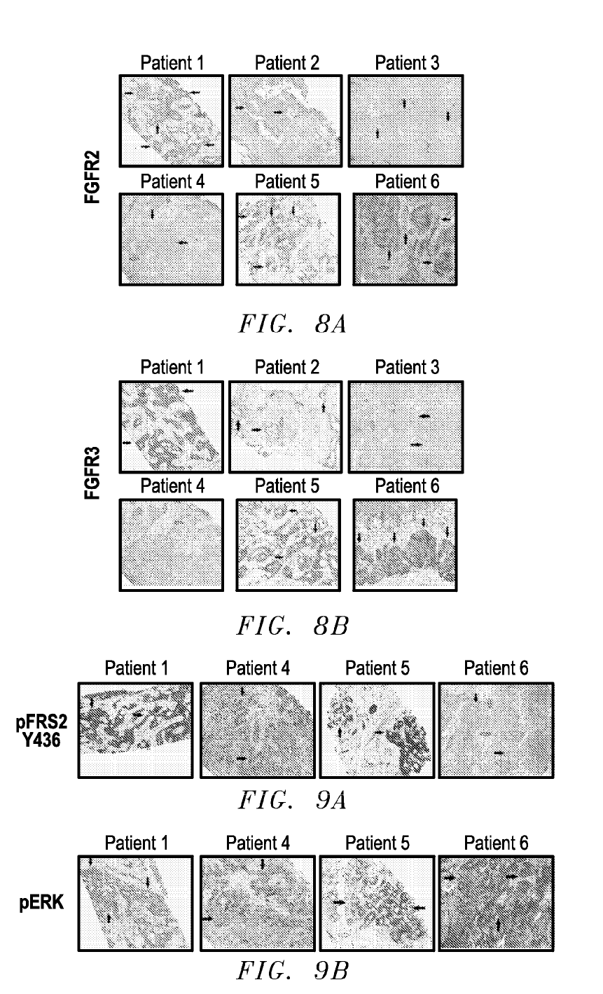

FIGS. 8A-8B are images of immunohistochemistry demonstrating FGFR2 and FGFR3 expression. FIG. 8A is an image of a tumor stained with FGFR2 antibody. Patient 1 demonstrates moderate cytoplasmic positivity (solid arrows); background fibro-inflammatory tissue is negative (empty arrows). Patient 2 demonstrates moderate cytoplasmic expression for FGFR2; tumor nuclei are negative. Patient 3 demonstrates tumor cells with negative nuclear and weak cytoplasmic expression of FGFR2 (solid arrows) with cells demonstrating moderate basolateral or complete membranous staining as well. Patient 4 demonstrates weak/moderate cytoplasmic positivity with multi-focal weak/moderate membranous expression (solid arrows); background fibro-inflammatory tissue demonstrates negative/weak staining (empty arrows). Patient 5 demonstrates weak/moderate cytoplasmic positivity with multi-focal moderate/strong membranous expression (solid arrows); background fibro-inflammatory tissue is negative (empty arrows). Patient 6 demonstrates moderate/strong cytoplasmic positivity (solid arrows); background lymphocytes are negative (empty arrows). FIG. 8B is an image of a tumor stained with FGFR3 antibody. Patient 1 demonstrates strong cytoplasmic positivity, variable nuclear expression and occasional moderate/strong membranous expression (solid arrows); background fibrous tissue is negative (empty arrows). Patient 2 demonstrates negatively staining background neutrophils (focally intraepithelial-far right) (empty arrows) and tumor cells with strong nuclear expression and moderate cytoplasmic positivity (solid arrows). Patient 3 demonstrates negatively staining background inflammation (empty arrows) and tumor cells with weak nuclear expression and moderate cytoplasmic positivity (solid arrows). Patient 4 demonstrates weak/moderate cytoplasmic positivity and variable nuclear expression; background fibro-inflammatory tissue demonstrates negative/weak positivity (empty arrows). Patient 5 demonstrates moderate cytoplasmic positivity, variable nuclear expression and strong multi-focal membranous expression (solid arrows); background fibrous tissue is negative. Patient 6 demonstrates diffuse/moderate/strong cytoplasmic and membranous positivity and variable nuclear expression (solid arrows); background lymphocytes are negative (empty arrows).

Patient 4 is a 62 year-old white female found to have a left-sided intrahepatic mass with satellite lesions, with metastasis to regional lymph nodes. Table 9 shows the clinical characteristics of 6 advanced, sporadic biliary tract cancer patients

TABLE 9

| | Patient 1 | Patient 2 | Patient 3 | Patient 4 | Patient 5 | Patient 6 |
|---|---|---|---|---|---|---|
| Age (years) | 64 | 66 | 50 | 62 | 50 | 43 |
| Gender | F | M | M | F | F | F |
| Location of | Intrahepatic | Intrahepatic/ | Intrahepatic | Intrahepatic | Intrahepatic | Intrahepatic |
| Primary Tumor | | Gallbladder | | | | |
| Stage | III | IV | IV | IV | IV | IV |
| CA19-9 | WNL | 1008 | WNL | WNL* | N/A | 56 |
| (Units/ml) | | | | | | |
| Sites of | N/A | Abdominal | Cervical, | Abdominal, | Liver, | Lungs |
| Metastasis | | Lymph | Thoracic, | Pelvic | Lungs, | |
| | | Nodes | Abdominal, | Lymph | Peritoneum | |
| | | | Pelvic | Nodes, | | |
| | | | Lymph Nodes | Liver | | |
| Underlying | Unknown | Unknown | Unknown | Unknown | Unknown | Unknown |
| Etiology | | | | | | |
| Liver fluke | No | No | No | No | No | No |
| Hepatitis B | Unknown | Unknown | Negative | Unknown | Unknown | Unknown |
| Hepatitis C | Unknown | Unknown | Negative | Unknown | Unknown | Unknown |
| Prior Surgical | No | Yes | Yes | No | Yes | No |
| Resection | | | | | | |
| Prior Radiation | No | No | No | No | No | No |
| Therapy | | | | | | |
| Systemic | Gem/Cis | Gem/Cis | Gem/Cis | Gem/Cis | Gem/Cis | Gem/Cis |
| Chemotherapy | | Capecitabine | | Gem/Cape | 5-FU/ | FOLFOX |
| | | | | PEGPH20 | Carbo | Pazopanib |
| Survival Status | Alive | Dead | Dead | Alive | Dead | Alive |
| Survival | 14.5+ | 8.8 | 9.0 | 9.3+ | 4.1 | 5.5+ |
| Duration from | | | | | | |
| biopsy | | | | | | |
| (months) | | | | | | |

F = female;
M = male;
WNL = Within Normal Limits;
Gem/Cis = Gemcitabine and Cisplatin;
Gem/Cape = Gemcitabine and Capecitabine;
PEGPH20 = pegylated hyaluronidase;
5-FU/Carbo = 5-Fluorouracil and Carboplatin;
FOLFOX - 5-FU, Leucovorin and Oxaliplatin,
*= WNL at baseline but 1408 U/ml prior to therapy and
N/A = Not Available

A biopsy of the liver mass revealed the presence of a poorly differentiated adenocarcinoma that was consistent with intrahepatic cholangiocarcinoma (CK7⁺, CEA⁺, CK20⁺, Hep-par 1⁻, TTF-1⁻). Table 10 shows the pathological characteristics of 6 advanced, sporadic biliary tract cancer patients.

TABLE 10

| | Patient 1 | Patient 2 | Patient 3 | Patient 4 | Patient 5 | Patient 6 |
|---|---|---|---|---|---|---|
| Grade/differentiation | Moderate | Moderate | Undifferentiated | Poor | Moderate | Poor |
| Biopsy Procedure | U/S | U/S | Excisional | U/S | U/S | Excisional |
| | Guided | Guided | Biopsy Lymph | Guided | Guided | Lung |
| | Liver | Liver | Node | Liver | Liver | Biopsy |
| | Biopsy | Biopsy | | Biopsy | Biopsy | |
| % Necrosis | 5 (1) | 0 (2) | 0-35 (7) | 0 (3) | 0-5 (3) | 0 |
| (aliquots) | | | | | | |
| % Tumor | 50 | 10-20 | 25-75 | 0-20 | 40-50 | 30 |
| % Stroma and | 50 | 80-90 | 25-75 | 80-100 | 50-60 | 70 |
| normal elements | | | | | | |
| Histological Type | NST** | NST | NST | NST | NST | NST |
| Clear Cell | No | No | No | No | No | No |
| Histology (Yes/No) | | | | | | |

U/S = Ultrasound
*NST: No special type.
**Rare gland formation with expression of cytokeratin, polyclonal CEA, and MOC-31. All were adenocarcinomas of no special types and high grades as defined by the World Health Organization Classification of Tumors of the Digestive System (Lyon 2000). Degree of differentiation is based on the percentage of glands (defined as having visible lumens by visual estimate) as follow: 95% or more glands-well differentiated, 40-94% glands-moderately differentiated, 5-39% glands-poorly differentiated, <5% glands-undifferentiated.

She received gemcitabine and cisplatin and obtained clinical benefit in the form of stable disease for 6 months, followed by disease progression. She was re-treated with gemcitabine and capecitabine systemic therapy and attained stable disease for 6 months, followed by disease progression. A clinical trial of pegylated hyaluronidase (PEGPH20) produced only stable disease for 4 months, followed again by disease progression. At this juncture, she underwent a liver biopsy to obtain tissue for whole genome characterization of her tumor.

TABLE 11

| | Fusion events | | | | | |
|---|---|---|---|---|---|---|
| | | | | | Predicted | |
| | | | Gene1 break | Gene2 break | Reciprocal | |
| | Gene1 | Gene2 | location | location | Translocation | Patient |
| Fusions | FGFR2 | MGEA5 | chr10:123243211 | chr10:103552699 | No | 4 |
| | FGFR2 | BICC1 | chr10:123237843 | chr10:60380614 | Yes | 5 |
| | BICC1 | FGFR2 | chr10:60272900 | chr10:123237848 | Yes | 5 |
| | FGFR2 | TACC3 | chr10:123243211 | chr4:1741428 | No | 6 |

Evaluation of pre-treatment immunohistochemistry demonstrated increased expression of FGFR2 and FGFR3 (FIG. 7) and Clinical Laboratory Improvement Amendments (CLIA) validation by quantitative PCR revealed increased expression of FGFR3. In order to further validate the activation of the receptor, we conducted immunohistochemistry (IHC) of pFRS2 Y436 and pERK(MAPK) that revealed strong expression of pFRS2 Y436 and pERK, thus confirming activation of the receptor.

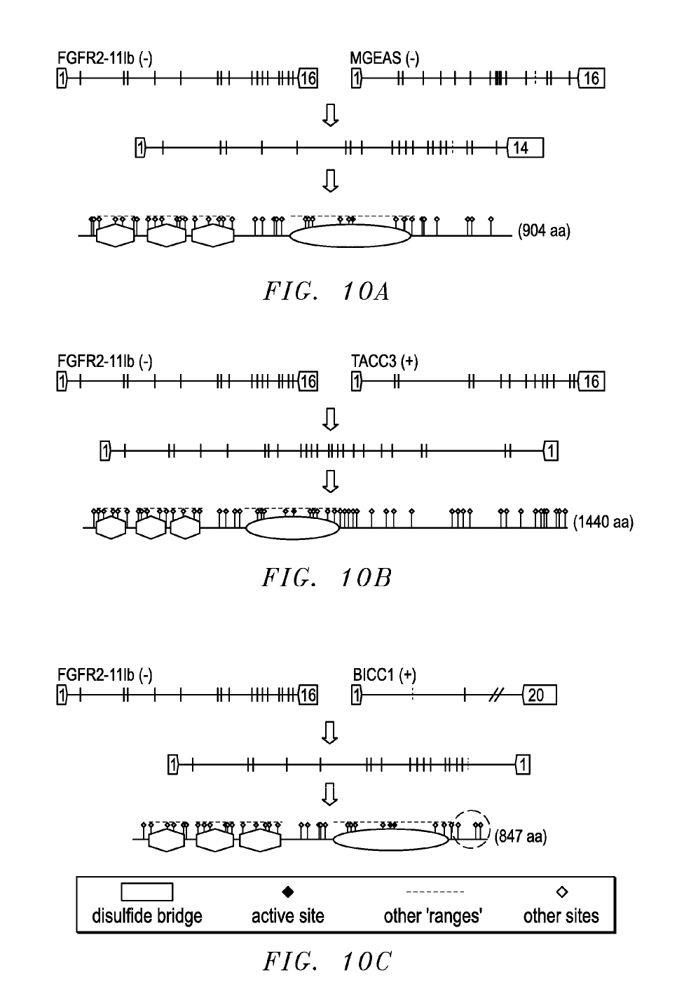

FIGS. 9A-9B are images showing immunohistochemistry demonstrating pFRS2 Y436, and pERK expression in Patients 1, 4, 5 and 6. FIG. 9A is an image showing a tumor stained with pFRS2 Y436 antibody. Patient 1 tumor cells demonstrating both strong cytoplasmic and nuclear expression of pFRS2 (solid arrows); background fibrous stroma is negative (empty arrows). Patient 4 tumor cells show strong nuclear expression and moderate to strong cytoplasmic positivity (solid arrows); occasional background fibrous stromal cells are negative for pFRS2 (empty arrows) and scattered tumor cells show basolateral/membranous staining as well (white arrows). Patient 5 tumor cells show intensely strong expression in both nuclei and cytoplasm (solid arrows); scattered background fibrous stromal cells are negative (empty arrows). Patient 6 tumor cells show negative nuclear expression of pFRS2, moderate cytoplasmic expression and basolateral or membranous expression of varying intensity (solid arrows); background fibrous stromal cells are negative (empty arrows). FIG. 9B is an image showing a tumor stained with pERK(MAPK) antibody. Patient 1 demonstrates negative/weak fibrous stroma (empty arrows) and tumor cells with negative nuclei and moderate to strong cytoplasmic expression (solid arrows). Patient 4 demonstrates negative inflammatory background (empty arrows) tumor cells with variable negative to strong nuclear expression and moderate to strong cytoplasmic positivity (solid arrows). Patient 5 demonstrates negative/weak fibrous stroma (empty arrows) and tumor cells with strong nuclear and cytoplasmic expression (solid arrows). Patient 6 demonstrates negative background lymphocytes/mononuclear inflammatory cells (empty arrows) and tumor cells with strong nuclear and cytoplasmic expression (solid arrows).

The FGFR2 fusion partner observed in this patient, MGEA5, is an enzyme responsible for the removal of O-GlcNAc from proteins [66]. Interestingly, soft tissue tumors myxoinflammatory fibroblastic sarcoma (MIFS) and hemosiderotic fibrolipomatous tumor (HFLT) both share a translocation event resulting in rearrangements in TGFBR3 and MGEA5 [67,68]. Associated with this translocation event is the upregulation of NPM3 and FGF8 [68], of which both genes are upregulated in this patient (fold change: NPM3=6.17865, FGF8=1.79769e+308). In breast cancer, grade III tumors had significantly lower MGEA5 expression than grade I tumors with a trend of decreasing expression observed with increasing tumor grade [66]. MGEA5 may play an important role in carcinogenesis as an FGFR fusion partner.

Patient 6 is a 43 year-old white female who underwent a right salpingo-oophorectomy and endometrial ablation in the context of a ruptured ovarian cyst (Table 9). Postoperatively she developed dyspnea and was found to have pulmonary nodules as well as a 5 cm left sided liver mass. Pathological evaluation of the liver mass was consistent with a moderately differentiated intrahepatic cholangiocarcinoma (CK7+, CK20−, TTF-1−) in the absence of any known risk factors (Table 10). She was treated systemically with gemcitabine and cisplatin and had stable disease for approximately 6 months, but was subsequently found to have disease progression. She was treated with FOLFOX for 7 months and again attained stable disease as best response to therapy but eventually experienced disease progression. Transcriptome analysis revealed the presence of an FGFR2-TACC3 fusion (Table 11). Further evaluation of phosphorylation of downstream targets FRS2 Y436, and ERK(MAPK) revealed strong expression of pERK and moderate expression of pFRS2 Y436 (FIG. 5), confirming activation of the receptor.

The FGFR2 fusion partner observed in this patient's tumor, TACC3, is overexpressed in many tumor types with enhanced cell proliferation, migration, and transformation observed in cells overexpressing TACC3 [70]. Furthermore regulation of ERK and PI3K/AKT by TACC3 may contribute in part to epithelial-mesenchymal transition (EMT) in cancer [70], a significant contributor to carcinogenesis.

Interestingly, TACC3 has been identified as a fusion partner to FGFR3 in bladder cancer, squamous cell lung cancer, oral cancer, head and neck cancer and glioblastoma multiforme [54].

TABLE 12

| Gene | Location | Mutation | CLIA report | Patient |
|---|---|---|---|---|
| ERRFI1 | chr1:8073509 | C/A | G/T - small | 3 |
| | | | percentage of T | |
| IDH2 | chr15:90631839 | T/A | A/T | 1 |
| NRAS | chr1:115258745 | C/G | C/G | 1 |
| PAK1 | chr11:77051696 | G/A | G/A | 2 |
| FGFR3 | Overexpressed | Fold change | qPCR fold | 4 |
| | | 3.32975 | difference = 2.13 | |
| FGFR3 | Overexpressed | Fold change | qPCR fold | 5 |
| | | 3.58524 | difference = 10.88 | |

Integrated analysis of sporadic intrahepatic cholangiocarcinoma (SIC) genomic and transcriptomic data led to the discovery of FGFR2 fusion products in three of six assessed patients. Members of the FGFR family (FGFR-1-4) have been associated with mutations, amplifications and translocation events with oncogenic potential [71]. FGFR fusions with oncogenic activity have been previously identified in bladder cancer (FGFR3) [72], lymphoma (FGFR1 and FGFR3) [73,74], acute myeloid leukemia (FGFR1) [75], multiple myeloma [76], myeloproliferative neoplasms [77], and most recently glioblastoma multiforme (FGFR1 and FGFR3) [78]. FGFR2, FGFR3 and FGFR4 have been found to be overexpressed in IDH1/IDH2 mutant biliary cancers [79], a context seen within Patient 1 in our study; although, no fusion events were depicted in these studies or in Patient 1. Table 13 shows differential gene expression of fibroblast growth factor receptor pathway family members in 5 patients with advanced sporadic biliary tract cancer.

TABLE 13

| | | Up/Down Regulated |
|---|---|---|
| | | (TumorVs.Normal), |
| Patients | FGF Family Member | Ordered by patient |
| 5 | FGFR1 | + |
| 1, 5 | FGFR2 | +/− |
| 1, 4, 5 | FGFR3 | +/+/+ |
| 1, 3, 4, 5 | FGFR4 | +/+/+/+ |
| 1, 4, 5 | FGF17 | +/+/+ |
| 1, 4* | FGFBP1 | +/+ |
| 1, 4* | FGF8 | +/+ |
| 1, 4, 5* | FGFR1OP | −/−/− |
| 1, 4, 5 | FGFBP3 | −/−/− |
| 1, 5 | FGF2 | −/−/− |
| 1, 4, 5 | FGF5 | −/−/− |
| 1, 4, 5 | FGF7 | −/−/− |
| 1, 4, 5 | FGF9 | −/−/− |
| 1, 4 | FGF10 | −/− |
| 3, 5 | FGF12 | +/− |
| 1, 4, 5 | FGF21 | −/−/− |
| | Transcripts that undergo changes | Up/Down Regulated |
| | in splicing during the E-M | (TumorVs.Normal), |
| Patients | Transition | Ordered by patient |
| 1, 4 | CTNND1 | −/− |
| 1, 4, 5 | CD44 | −/−/− |
| | mRNA splicing factor that | Up/Down Regulated |
| | regulates the formation of | (TumorVs.Normal), |
| Patients | epithelial cell-specific isoforms | Ordered by patient |
| 1, 4, 5 | ESRP1 | +/+/+ |

*Not significant (10⁻³ is threshold of significance)

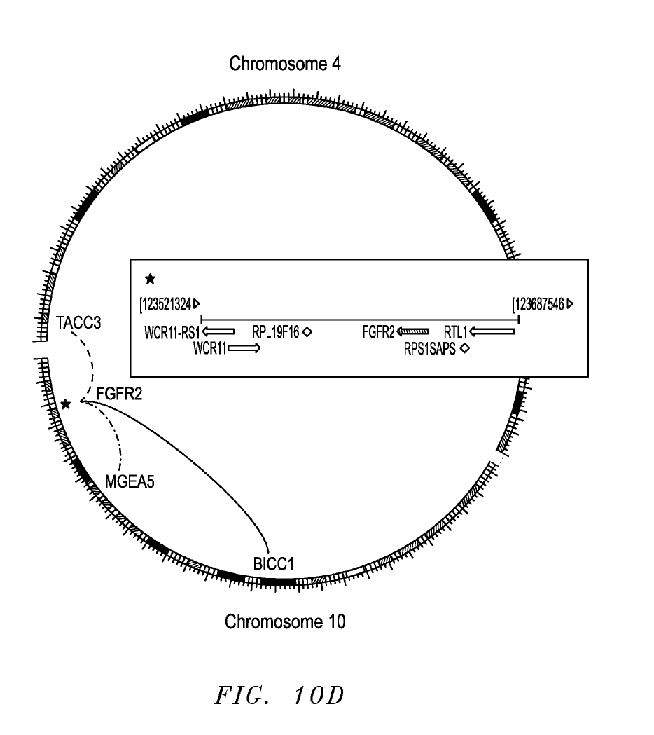

FIG. 10A-10D are images showing transcripts and hypothetical protein products modeled to illustrate the potential functional impact of fusion events involving FGFR2. FIG. 10A shows the FGFR2 fusion event involving MGEA5 (Patient 4) and FIG. 10C shows the FGFR2 fusion event involving BICC1 (Patient 5, reciprocal event). FIG. 10D shows interchromosomal fusion events. In addition, Patient 6 carried an interchromosomal fusion event involving FGFR2 and TACC3 FIG. 10B. The FGFR2 gene encodes for several isoforms with eleven representative transcripts and Patients 4, 5, and 6 carry fusions involving the epithelial cell specific transcript isoform (FGFR2 -IIIb). All identified fusion breakpoints are close in proximity and are predicted to occur within the last intron of the transcript and terminal to a known protein tyrosine kinase domain (FIGS. 10A-10C, gold domain). Predicted “Other” sites for all of the fusion protein models are the same and include the following: Casein kinase II phosphorylation sites, N-glycosylation sites, Protein kinase C phosphorylation sites, N-myristoylation sites, Tyrosine kinase phosphorylation sites, and cAMP-/cGMP-dependent protein kinase phosphorylation sites (FIGS. 10A-10C, grey triangle annotations). In all cases, fusions result in a predicted expansion of Casein kinase II phosphorylation and Protein kinase C phosphorylation sites. A protein product model is shown only for one of the reciprocal events involving the FGFR2 and BICC1 genes (FGFR2→BICC1, FIG. 10C). The fusion breakpoints of the reciprocal events effect Exons 1 and 2 of the BICC1 gene, which translates to a difference of a predicted phosphoserine site within the Casein kinase II phosphorylation region (FIG. 10C, purple triangle within red circle). The FGFR2 gene is located within a fragile site region (FRA10F) and is flanked by two ribosomal protein pseudogenes, RPS15AP5 and RPL19P16 (see D inset (*)), whose repetitive sequence content may also contribute to genomic instability at the FGFR2 initiation site.

Although the gene partner fused to FGFR2 was different for each Patient (MGEA5, BICC1 and TACC3), the breakpoints in FGFR2 all occurred within the last intron distal to the last coding exon and terminal protein tyrosine kinase domain. All three fusions were validated at the DNA and/or RNA level of FGFR2 (Table 13) fusions in 3 Patients with advanced sporadic biliary tract cancer.

TABLE 14

| | Annealing | PCR | | SEQ | |
|---|---|---|---|---|---|
| Fusion | site | input | Dir | ID NO | Primer sequence |
| FGFR2-MGEA5 | FGFR2 | gDNA | F | 1 | 5′-CTGACTATAACCACGTACCC-3′ |
| | MGEA5 | gDNA | R | 2 | 5′-AGGGAGAAATTAAAGAACTTGG-3′ |
| | FGFR2 | cDNA | F | 3 | 5′-TGATGATGAGGGACTGTTG-3′ |
| | MGEA5 | cDNA | R | 4 | 5′-GAGTTCCTTGTCACCATTTG-3′ |
| FGFF2-BICC1 | FGFR2 | gDNA | F | 5 | 5′-GGCAGAAGAAGAAAGTTGG-3′ |
| | BICC1 | gDNA | R | 6 | 5′-ACTACTGCAGTTTGTTCAAT-3′ |
| | FGFR2 | cDNA | F | 7 | 5′-TGATGATGAGGGACTGTTG-3′ |
| | BICC1 | cDNA | R | 8 | 5′-TGTGTGCTCACAGGAATAG-3′ |
| BICC1-FGFR2 | BICC1 | cDNA | F | 9 | 5′-CGTGGACAGGAAGAAACT-3′ |
| | FGFR2 | cDNA | R | 10 | 5′-GTGTGGATACTGAGGAAG-3′ |
| FGFR2-TACC3 | FGFR2 | gDNA | F | 11 | 5′-TGACCCCCTAATCTAGTTGC-3′ |
| | TACC3 | gDNA | R | 12 | 5′-AACCTGTCCATGATCTTCCT-3′ |

Amongst these fusions, the FGFR2-BICC1 fusion has recently been independently identified in SIC [53,54]. For this particular fusion product we observed, and validated, the presence of two fusion isoforms (FGFR2-BICC1 and BICC1-FGFR2). Interestingly, BICC1 is a negative regulator of Wnt signaling [80] and when comparing expression of tumor and normal tissue we observed differentially expressed Wnt signaling genes, APC (fold change −4.75027), GSK3B (fold change −3.35309), and CTNNB1 (fold change −1.73148), yet when the expression was compared to other cholangiocarincomas, no difference was observed.

The FGFR genes encode multiple structural variants through alternative splicing. Notably, RNASeq data revealed that the FGFR2-IIIb isoform was present in all fusions detected in our study and has been shown to have selectivity for epithelial cells as opposed to the FGFR2-IIIc isoform, which is found selectively in mesenchymal cells [81]. Paradoxically, wildtype FGFR2-IIIb has been described as a tumor suppressor in pre-clinical systems of bladder cancer and prostate cancer [82,83]. As such, FGFR signaling appears context-dependent and exhibits variability in disparate tumor types.

Comparative pathway analysis of genes carrying mutations/aberrant in copy number identified additional potential therapeutic targets belonging to, or intimately integrated with, the EGFR and FGFR signaling pathways (FIG. 6, Tables 5-7). Interestingly, most of these pathways also have known roles in mediating epithelial-to-mesenchymal cell transitions, which occur frequently during development as well as during tumorigenesis [60]. Patients 3 and 4 harbored aberrations in several genes acting in cadherin signaling pathways (Tables 6-7), which are important for maintaining cell-cell adhesion [63].

Whole genome sequencing for Patients 1, 3, 4, and 5. 1.1 μg genomic DNA was used to generate separate long insert whole genome libraries for each sample using Illumina's (San Diego, Calif.) TruSeq DNA Sample Prep Kit (catalog #FC-121-2001). In summary, genomic DNAs are fragmented to a target size of 900-1000 bp on the Covaris E210. 100 ng of the sample was run on a 1% TAE gel to verify fragmentation. Samples were end repaired and purified with Ampure XP beads using a 1:1 bead volume to sample volume ratio, and ligated with indexed adapters. Samples are size selected at approximately 1000 bp by running samples on a 1.5% TAE gel and purified using Bio-Rad Freeze 'n Squeeze columns and Ampure XP beads. Size selected products are then amplified using PCR and products were cleaned using Ampure XP beads. Whole genome sequencing for Patient 2. 300 ng genomic tumor and normal DNA was used to create whole genome libraries. Samples were fragmented on the Covaris E210 to a target size of 200-300 bp and 50 ng of the fragmented product was run on a 2% TAE gel to verify fragmentation. Whole genome libraries were prepared using Illumina's TruSeq DNA Sample Prep Kit.

Exome sequencing for Patients 1 and 3. 1.1μg genomic DNA for each sample was fragmented to a target size of 150-200 bp on the Covaris E210. 100 ng of fragmented product was run on TAE gel to verify fragmentation. The remaining 1 μg of fragmented DNA was prepared using Agilent's SureSelect^{XT} and SureSelect^{XT} Human All Exon 50 Mb kit (catalog #G7544C). Exome sequencing for Patient 2. 50ng genomic tumor and normal DNA was used to create exome libraries using Illumina's Nextera Exome Enrichment kit (catalog #FC-121-1204) following the manufacturer's protocol. Exome sequencing for Patients 4 and 5. 1 μg of each tumor and germline DNA sample was used to generate separate exome libraries. Libraries were prepared using Illumina's TruSeq DNA Sample Prep Kit and Exome Enrichment Kit (catalog #FC-121-1008) following the manufacturer's protocols. Exome sequencing for Patient 6. 3 μg of genomic tumor and normal DNA was fragmented on the Covaris E210 to a target size of 150-200 bp. Exome libraries were prepared with Agilent's (Santa Clara, Calif.) SureSelectXT Human All Exon V4 library preparation kit (catalog #5190-4632) and SureSelectXT Human All Exon V4+UTRs (catalog #5190-4637) following the manufacturer's protocols.

RNA sequencing for Patients 1, 2 and 3. 50 ng total RNA was used to generate whole transcriptome libraries for RNA sequencing. Using the Nugen Ovation RNA-Seq System v2 (catalog #7102), total RNA was used to generate double stranded cDNA, which was subsequently amplified using Nugen's SPIA linear amplification process. Amplified products were cleaned using Qiagen's QIAquick PCR Purification Kit and quantitated using Invitrogen's Quant-iT Picogreen. 1 μg of amplified cDNA was fragmented on the Covaris E210 to a target size of 300 bp. Illumina's TruSeq DNA Sample Preparation Kit was used to prepare libraries from 1 μg amplified cDNA.

RNA sequencing for Patients 4, 5 and 6. 1μg of total RNA for each sample was used to generate RNA sequencing libraries using Illumina's TruSeq RNA Sample Prep Kit V2 (catalog #RS-122-2001) following the manufacturer's protocol.

Paired End Sequencing. Libraries with a 1% phiX spike-in were used to generate clusters on HiSeq Paired End v3 flowcells on the Illumina cBot using Illumina's TruSeq PE Cluster Kit v3 (catalog #PE-401-3001). Clustered flowcells were sequenced by synthesis on the Illumina HiSeq 2000 using paired-end technology and Illumina's TruSeq SBS Kit.

Alignment and variant calling for whole genome and whole exome. For whole genome and exome sequencing fastq files were aligned with BWA 0.6.2 to GRCh37.62 and the SAM output were converted to a sorted BAM file using SAMtools 0.1.18. BAM files were then processed through indel realignment, mark duplicates, and recalibration steps in this order with GATK 1.5 where dpsnp135 was used for known SNPs and 1000 Genomes' ALL.wgs.low_coverage_vqsr.20101123 was used for known indels. Lane level sample BAMs were then merged with Picard 1.65 if they were sequenced across multiple lanes. Comparative variant calling for exome data was conducted with Seurat [105].

Previously described copy number and translocation detection were applied to the whole genome long insert sequencing data [59]. Copy number detection was based on a log 2 comparison of normalized physical coverage (or clonal coverage) across tumor and normal whole genome long-insert sequencing data, where physical coverage was calculated by considering the entire region a paired-end fragment spans on the genome, then the coverage at 100 bp intervals was kept. Normal and tumor physical coverage was then normalized, smoothed and filtered for highly repetitive regions prior to calculating the log 2 comparison. Translocation detection was based on discordant read evidence in the tumor whole genome sequencing data compared to its corresponding normal data. In order for the structural variant to be called there needs to be greater than 7 read pairs mapping to both sides of the breakpoint. The unique feature of the long-insert whole-genome sequencing was the long overall fragment size (˜1 kb), where by two 100 bp reads flank a region of ˜800 bp. The separation of forward and reverse reads increases the overall probability that the read pairs do not cross the breakpoint and confound mapping.

For RNA sequencing, lane level fastq files were appended together if they were across multiple lanes. These fastq files were then aligned with TopHat 2.0.6 to GRCh37.62 using ensemb1.63.genes.gtf as GTF file. Changes in transcript expression were calculated with Cuffdiff 2.0.2. For novel fusion discovery reads were aligned with TopHat-Fusion 2.0.6 [106] (Patients 2, 3, 4 and 6). In addition, Chimerascan 0.4.5 [107] was used to detect fusions in Patient 1, deFuse 5.0 [108] used in Patients 2, 3 and 5 and SnowShoes [109] for Patients 2 and 5.

Briefly, slides were dewaxed, rehydrated and antigen retrieved on-line on the BONDMAX™ autostainer (Leica Microsystems, INC Bannockburn, Ill.). Slides were then subjected to heat-induced epitope retrieval using a proprietary EDTA-based retrieval solution. Endogenous peroxidase was then blocked and slides were incubated with the following antibodies: FGFR2 (BEK, Santa Cruz, catalog #sc-20735), FGFR3 (C-15, Santa Cruz, catalog #sc-123), panAKT (Cell Signaling Technology, catalog #4685, pAKT (Cell Signaling Technology, catalog #4060), EGFR (Cell Signaling Technology, catalog #4267, pEGFR (Cell Signaling Technology, catalog #2234), MAPK/ERK1/2 (Cell Signaling Technology, catalog #4695), pMAPK/pERK (Cell Signaling Technology, catalog #4376) and pFRS2 Y436 (Abcam, catalog #ab78195). Sections were visualized using the Polymer Refine Detection kit (Leica) using diaminobenzidine chromogen as substrate.

Fluorescent in-situ hybridization (FISH) was performed on formalin-fixed paraffin-embedded (FFPE) specimens using standard protocols and dual-color break-apart rearrangement probes specific to the FGFR2 gene (Abbott Molecular, Inc. Des Plaines, Ill.) located at 10 q26. The 5′ FGFR2 signal was labeled with Spectrum Orange (orange) and the 3′ FGFR2 signal was labeled with Spectrum Green (green).

In some embodiments, homology, sequence identity or complementarity, is between the antisense compound and target is from about 40% to about 60%. In some embodiments, homology, sequence identity or complementarity, is from about 60% to about 70%. In some embodiments, homology, sequence identity or complementarity, is from about 70% to about 80%. In some embodiments, homology, sequence identity or complementarity, is from about 80% to about 90%. In some embodiments, homology, sequence identity or complementarity, is about 90%, about 92%, about 94%, about 95%, about 96%, about 97%, about 98%, about 99% or about 100%.

Examples of cancers (including solid tumors) which may be treated (or inhibited) include, but are not limited to, a carcinoma, for example a carcinoma of the bladder, breast, colon (e.g. colorectal carcinomas such as colon adenocarcinoma and colon adenoma), kidney, epidermis, liver, lung, for example adenocarcinoma, small cell lung cancer and non-small cell lung carcinomas, oesophagus, gall bladder, ovary, pancreas e.g. exocrine pancreatic carcinoma, stomach, cervix, endometrium, thyroid, prostate, or skin, for example squamous cell carcinoma; a hematopoietic tumour of lymphoid lineage, for example leukemia, acute lymphocytic leukemia, chronic lymphocytic leukemia, B-cell lymphoma, T-cell lymphoma, Hodgkin's lymphoma, non-Hodgkin's lymphoma, hairy cell lymphoma, or Burkett's lymphoma; a hematopoietic tumour of myeloid lineage, for example leukemias, acute and chronic myelogenous leukemias, myeloproliferative syndrome, myelodysplastic syndrome, or promyelocytic leukemia; multiple myeloma; thyroid follicular cancer; a tumour of mesenchymal origin, for example fibrosarcoma or rhabdomyosarcoma; a tumour of the central or peripheral nervous system, for example astrocytoma, neuroblastoma, glioma or schwannoma; melanoma; seminoma; teratocarcinoma; osteosarcoma; xeroderma pigmentosum; keratoctanthoma; thyroid follicular cancer; or Kaposi's sarcoma.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

1. A method of characterizing a cancer comprising the steps of
(a) obtaining a sample from a subject suspected of having cancer; and
(b) determining whether a fibroblast growth factor receptor (FGFR) fusion is present in the sample, wherein the FGFR fusion comprises a FGFR locus, thereby characterizing the cancer based on the presence or absence of the FGFR fusion.

2. The method of claim 1, wherein the presence of the FGFR fusion is determined in a break apart fluorescence in situ hybridization (FISH) assay.

3. The method of claim 1 wherein the FGFR fusion includes AHCYL1, BICC1, BUB1, CDCA8, DNAH5, MGEA5, TACC1, or TACC3.

4. The method of claim 1, wherein the presence of the FGFR fusion is determined by the binding of a first probe that binds to a FGFR2 breakpoint.

5. The method of claim 4, wherein the first probe has between 90-99% homology to the FGFR2 breakpoint.

6. The method of claim 4, wherein the presence of the FGFR fusion is determined by the binding of a second probe that binds to a second breakpoint.

7. The method of claim 4, wherein the second probe has between 90-99% homology to the second breakpoint.

8. (canceled)

9. The method of claim 1 wherein the cancer comprises a carcinoma of the bladder, breast, colon, kidney, epidermis, liver, lung, esophagus, gall bladder, ovary, pancreas, stomach, cervix, endometrium, thyroid, prostate, skin, a hematopoietic tumour of lymphoid lineage, a hematopoietic tumour of myeloid lineage, multiple myeloma, thyroid follicular cancer, a tumour of mesenchymal origin, a tumour of the central or peripheral nervous system, seminoma, teratocarcinoma, osteosarcoma, xeroderma pigmentosum, keratoctanthoma, thyroid follicular cancer, Kaposi's sarcoma or a sporadic intrahepatic cholangiocarcinoma.

10. (canceled)

11. A method for detecting a fibroblast growth factor receptor (FGFR) translocation event in one or more cancer cells comprising the steps of:
contacting a sample suspected of comprising one or more cancer cells with a plurality of distinguishably labeled probes capable of hybridizing to a portion of a fibroblast growth factor receptor (FGFR) fusion in the one or more cancer cells;
hybridizing a first probe to a first region to form a first hybridization complex;
hybridizing a second probe to a second region to form a second hybridization complex; and
analyzing the first hybridization complex and the second hybridization complex to identify the presence of a FGFR fusion.

12. The method of claim 11, wherein the presence of the FGFR fusion is determined in a fluorescence in situ hybridization (FISH) assay.

13. The method of claim 11, wherein the FGFR fusion includes AHCYL1, BICC1, BUB1, CDCA8, DNAH5, MGEA5, TACC1, or TACC3.

14. The method of claim 13, wherein the first probe hybridizes to a FGFR region and the second probe hybridizes to a region including AHCYL1, BICC1, BUB 1, CDCA8, DNAH5, MGEA5, TACC1, or TACC3.

15. (canceled)

16. The method of claim 11, wherein the presence of the FGFR fusion is determined by the binding of a first probe that binds to a FGFR2 breakpoint.

17. The method of claim 11, wherein the presence of the FGFR fusion is determined by the binding of a second probe that binds to a second breakpoint.

18. The method of claim 17, wherein the first probe has between 90-99% homology to the FGFR2 breakpoint and the second probe has between 90-99% homology to the second breakpoint.

19. (canceled)

20. The method of claim 11, wherein the cancer is a sporadic intrahepatic cholangiocarcinoma.

21. A method for identifying the response of a proliferative disorder responsive to treatment comprising the steps of:
detecting one or more FGFR biomarkers selected for a FGFR fusion that is indicative of the prognosis of a subject.

22. The method of claim 21 wherein the one or more FGFR translocation events comprises a FGFR locus.

23. (canceled)

24. The method of claim 21 wherein the translocation events is a FGFR fusion comprising one or more selected from AHCYL1, BICC1, BUB1, CDCA8, DNAH5, MGEA5, TACC1, and TACC3.

25. The method of claim 21 wherein the translocation events is a FGFR fusion comprising a FGFR2-MGEA5 fusion, a FGFR2-TACC3 fusion, or a FGFR2-BICC1 fusion.
